SEQUENCE LISTING (30 sequences)

SEQ ID NO: 1 - DNA, 19 bp, Artificial Sequence
  oligonucleotides
aacaagcatccatggacct

SEQ ID NO: 2 - DNA, 19 bp, Artificial Sequence
  oligonucleotides
ctctgggaaccctttccag

SEQ ID NO: 3 - DNA, 20 bp, Artificial Sequence
  oligonucleotides
acctcacccctcagtccaaa

SEQ ID NO: 4 - DNA, 19 bp, Artificial Sequence
  oligonucleotides
tggtcggtcccaggatctg

SEQ ID NO: 5 - DNA, 22 bp, Artificial Sequence
  oligonucleotides
atggacgggactattaaggagg

SEQ ID NO: 6 - DNA, 22 bp, Artificial Sequence
  oligonucleotides
gaagcagtcatatctgccttgg

SEQ ID NO: 7 - DNA, 19 bp, Artificial Sequence
  oligonucleotides
cactgctttgggagccttc

SEQ ID NO: 8 - DNA, 20 bp, Artificial Sequence
  oligonucleotides
gggcagcgattcatttttct

SEQ ID NO: 9 - DNA, 21 bp, Artificial Sequence
  oligonucleotides
ctgccagtccgaaaatggaac

SEQ ID NO: 10 - DNA, 20 bp, Artificial Sequence
  oligonucleotides
cttcatccaccggggctatc

SEQ ID NO: 11 - DNA, 23 bp, Artificial Sequence
  oligonucleotides
tttggcaaatacaacccttcaga

SEQ ID NO: 12 - DNA, 21 bp, Artificial Sequence
  oligonucleotides
gcagaagatactgtcaccacc

SEQ ID NO: 13 - DNA, 19 bp, Artificial Sequence
  oligonucleotides
atgcctcgcgctttctctc

SEQ ID NO: 14 - DNA, 18 bp, Artificial Sequence
  oligonucleotides
gtagtcccgctgacagta

SEQ ID NO: 15 - DNA, 21 bp, Artificial Sequence
  oligonucleotides
atgtggaagtcgagaggcgat

SEQ ID NO: 16 - DNA, 23 bp, Artificial Sequence
  oligonucleotides
cgaatagccatccactattgtcc

SEQ ID NO: 17 - DNA, 21 bp, Artificial Sequence
  oligonucleotides
gtcccagacatcagggagtaa

SEQ ID NO: 18 - DNA, 21 bp, Artificial Sequence
  oligonucleotides
tcggatacttcagcgtcagga

SEQ ID NO: 19 - DNA, 24 bp, Artificial Sequence
  oligonucleotides
aaatggtgaaggtcggtgtgaacg

SEQ ID NO: 20 - DNA, 24 bp, Artificial Sequence
  oligonucleotides
ggtcaatgaaggggtcgttgatgg

SEQ ID NO: 21 - DNA, 21 bp, Artificial Sequence
  oligonucleotides
gacagcgacaagattccgttc

SEQ ID NO: 22 - DNA, 19 bp, Artificial Sequence
  oligonucleotides
ggtgcccgacttcaccttc

SEQ ID NO: 23 - DNA, 21 bp, Artificial Sequence
  oligonucleotides
atgctgggaaagtcatggaag

SEQ ID NO: 24 - DNA, 20 bp, Artificial Sequence
  oligonucleotides
cgtgttctggtcacgagaga

SEQ ID NO: 25 - DNA, 20 bp, Artificial Sequence
  oligonucleotides
agtacccgcatctgcacaac

SEQ ID NO: 26 - DNA, 20 bp, Artificial Sequence
  oligonucleotides
acgaagggtctcttctcgct

SEQ ID NO: 27 - DNA, 19 bp, Artificial Sequence
  oligonucleotides
gatgcgggatacgccagtg

SEQ ID NO: 28 - DNA, 21 bp, Artificial Sequence
  oligonucleotides
ccacctcgcctttcaccttta

SEQ ID NO: 29 - DNA, 19 bp, Artificial Sequence
  oligonucleotides
cctgtcaccaacgtctcgc

SEQ ID NO: 30 - DNA, 19 bp, Artificial Sequence
  oligonucleotides
gcaactcgtcggcagtttg

(19) United States
(12) Patent Application Publication
(10) Pub. No.: US 2019/0352601 A1
(43) Pub. Date: Nov. 21, 2019

(54) ENGINEERING BLOOD VESSEL CELLS FOR TRANSPLANTATION

(71) Applicant: CORNELL UNIVERSITY, Ithaca, NY (US)

(72) Inventors: Shahin RAFII, Ithaca, NY (US); Will SCHACHTERLE, Ithaca, NY (US)

(21) Appl. No.: 16/482,420

(22) PCT Filed: Feb. 1, 2018
(86) PCT No.: PCT/US2018/016445

§ 371 Date: Jul. 31, 2019

Related U.S. Application Data
(60) Provisional application No. 62/453,106, filed on Feb. 1, 2017.

(51) Int. Cl. C12N 5/071; C12N 15/85; A61P 17/02; A61P 35/00; A61K 35/44

(52) U.S. Cl. CPC C12N 5/069; C12N 15/85; A61P 17/02; C12N 2506/025; A61K 35/44; C12N 2501/998; C12N 2501/999; A61P 35/00

(57) ABSTRACT

This disclosure is directed to methods for reproducibly generating substantial amounts of endothelial cells from non-vascular cells that display improved functionality and engraftability. The endothelial cells generated in accordance with the present methodology, as well as therapeutic methods utilizing these cells, are also disclosed.

### CROSS REFERENCE TO RELATED APPLICATION

This application claims the benefit of priority from U.S. Provisional Application No. 62/453,106, filed Feb. 1, 2017, the entire contents of which are incorporated herein by reference.

### INCORPORATION BY REFERENCE OF SEQUENCE LISTING

The Sequence Listing in an ASCII text file, named as 34635_SEQ_ST25.txt of 6 KB, created on Jan. 24, 2018, and submitted to the United States Patent and Trademark Office via EFS-Web, is incorporated herein by reference.

### FIELD OF THE DISCLOSURE

This disclosure relates to methods for generating functional and durable endothelial cells with improved functionality and engraftability. In particular, this invention relates to generation of substantial amounts of bona fide endothelial cells from non-vascular cells by reprogramming the non-vascular cells through enforced expression of ETS-TFs and Sox17 in conjunction with suppression of the TGFβ signaling pathway.

### BACKGROUND

For many traumatic and ischemic vascular diseases, transplanting non-lymphatic blood vessel endothelial cells (ECs) is an attractive regenerative therapy because vascularized and functional ECs support the metabolic needs of damaged tissues (Ramasamy, S. K. et al., Trends Cell Biol, 25, 148-157, (2015); Gulati, R. & Simari, R. D., Dis Model Mech, 2, 130-137, (2009)) and choreograph tissue regrowth via perfusion-independent angiocrine functions (Ding, B. S. et al. Endothelial-derived angiocrine signals induce and sustain regenerative lung alveolarization. Cell 147, 539-553, (2011); Takebe, T. et al., Nature, 499, 481-484, (2013); Hu, J. et al., Science. 343, 416-419, (2014); Poulos, M. G. et al., Stem Cell Reports, (2015), Rafii, S. et al, Nature, 529, 316-325, (2016)). However, assessing EC transplantation therapies is made impractical by the difficulty of purifying and expanding primary ECs that sustain their vascular fate during in vitro cultivation. A large number of stable vascular cells are necessary for testing revascularizing and regenerative EC therapies. Cellular reprogramming is appealing because it provides a therapeutically-relevant path for converting an available cell type to a transplantable EC with regenerative capabilities. While angiogenic factors can coax non-vascular cells into EC-like lineages (Margariti, A. et al., PNAS,109, 13793-13798, (2012); Li, J. et al., Arteriosclerosis, thrombosis, and vascular biology, 33, 1366-1375, (2013); Prasain, N. et al. Nature Biotechnology, 32, 1151-1157, (2014); Israely, E. et al. Stem Cells, 32, 177-190, (2014); Kurian, L. et al., Nat Methods, 10, 77-83, (2013)), the factors that drive the EC properties required to meet the translational goals of tissue regeneration have been difficult to dissociate from principal lineage specification.

Re-supply of oxygen and nutrients via blood vessels is an attractive therapy for pathologies that arise from diseased or insufficient vasculature. Blood vessels are composed of ECs that line vessels and interface between blood and surrounding tissue. Positioned as such, ECs also provide powerful paracrine, or angiocrine, signals to parenchymal cells that govern their homeostatic and regenerative status. Therefore, ECs that could be delivered via local or circulatory injection could serve as therapies in diverse therapeutic contexts such as radiation countermeasures and diabetes.

To uncover factors necessary for conversion of non-vascular cells into transplantable blood vessel ECs that engraft, it is necessary to identify permissive mesenchymal or epithelial cells that are amenable to conversion into the EC identity. Pluripotent stem cells differentiate into ECs but this process is driven by pre-determined programs that can be challenging to tease apart by reductive approaches. Also, ECs generated by pluripotent cells can be unstable, multipotent, and/or immature (Israely, E. et al. Stem Cells, 32, 177-190, (2014); Kurian, L. et al., Nat Methods, 10, 77-83, (2013); McCloskey, K. E. et al., J Vasc Res, 43, 411-421, (2006); Kurian, L. et al. Nat Methods, 10, 77-83, (2013)). Human amniotic fluid cells can be converted to vascular endothelial cells (RACVECs, reprogrammed amniotic cells to vascular endothelial cells) by overexpressing the Ets transcription factors (TFs) Etv2, Fli1, and Erg while also inhibiting TGF-β signaling (Ginsberg, M. et al., Cell, 151, 559-575, (2012); also described in U.S. Pat. No. 9,637,723, which is incorporated by reference in its entirety) Amniotic cells, unlike pluripotent cells, are terminally differentiated, non-vascular, parenchymal cells yet they appear to retain some developmental plasticity Amniotic cells are appealing because they are routinely obtained from pregnant subjects with broad genetic and ethnic backgrounds (Murphy, S. V. & Atala, A., Semin Reprod Med, 31, 62-68, (2013)).

Xenobiotic barriers impede thorough functional testing and direct comparison of human RACVECs to adult ECs. To facilitate in vivo functional testing of converted cells, the inventors searched for murine cell sources that are accessible and amenable to EC conversion. Converted mouse amniotic cells, or murine RACVECs (subsequently be referred to as simply, “RACVECs”), stably adopted an EC-like immunophenotype and acquired a transcriptome highly similar to cultured adult ECs. Despite their stable EC-like identity, murine RACVECs performed poorly in tests of EC function compared to cultured adult ECs. To identify mechanisms that might drive functional engraftment of RACVEC into host vasculature, we utilized constitutively active Akt-signaling. Active Akt-signaling is detectable in most normal adult EC beds (Lassoued, W. et al., Cancer Biol Ther, 10, 1326-1333 (2010)) and enforced constitutive Akt-signaling enables survival of cultured ES-derived and adult ECs, likely by emulating in vivo EC microenvironment cues such as tuned growth factor signals, cell-cell contacts, and shear forces (Dimmeler, S. et al., Circulation Res., 83, 334-341 (1998); Dimmeler, S. et al. Nature, 399, 601-605, (1999); Fujio, Y. & Walsh, K., JBC, 274, 16349-16354 (1999)). Akt-signaling rescued the functional deficiencies of RACVECs by activating EC morphogenesis genes, including Sox17, and refining the Fli1 genomic binding site purview to enrich for Fli1 sites common to primary ECs including those near Sox consensus sequences. Enforced expression of Sox17 in RACVECs enabled converted cells to form stable vascular networks in vitro and in vivo and obviated the need for constitutive Akt-signaling.

Therapeutic delivery of ECs by transplantation requires that the cells 1. can be stably expanded to clinically-relevant numbers, 2. incorporate into extant vessels, and 3. compose neo-vessels. There are several strategies to engineer ECs that meet these goals, but they fail one or more of these pre-requisites. Adult ECs directly isolated from donors do not stably expand. Circulating endothelial cells, derived from bone marrow, similarly expand poorly in vitro, and their identification remains controversial. Directed differentiation of pluripotent cells generates large numbers of ECs but they tend to lose their endothelial identity as they expand. In contrast, direct conversion of readily available differentiated cells generates large numbers of stable ECs without any further modification.

ECs derived from adult tissue, pluripotent sources, and direct conversion can be stably propagated by the addition of a constitutively active myristoylated Akt molecule (myr-Akt). Additionally myr-Akt ECs engraft into tissue after transplantation. However, transplantation of cells with constitutive Akt signaling is problematic because of Akt's well-established role in oncogenesis. The inventors of this disclosure compared converted cells derived from mouse amniotic fluid with and without myr-Akt and identified the transcription factor Sox17 as being downstream of Akt. Enforced expression of Sox17, instead of myr-Akt, endowed converted cells with the ability to stably and functionally engraft after transplantation.

### SUMMARY OF THE DISCLOSURE

In one aspect, this disclosure provides a method of providing endothelial cells, comprising expressing transcription factor Sox17 from an exogenous nucleic acid in reprogramming-derived endothelial cells (rECs).

In some embodiments, the rECs are characterized by expression of surface markers, VE-cadherin, CD31 and VEGFR2, wherein the rECs comprise an exogenously introduced nucleic acid encoding FLI1.

In some embodiments, the rECs are transduced with a vector comprising a nucleic acid encoding Sox17 to achieve expression of Sox17. In some embodiments, an mRNA encoding Sox17 are delivered into rECs to achieve expression of Sox17. In some embodiments, Sox17 is expressed constitutively for at least 20 days. In some embodiments, the Sox17-rECs are cultured for a total duration of at least 28 days. In some embodiments, the Sox17-rECs are cultured for a total duration of at least 42 days.

In some embodiments, the rECs are derived from non-vascular cells by a process comprising expressing transcription factors ETV2, FLI1 and ERG from exogenous nucleic acids in the non-vascular cells in the presence of a TGFβ signaling inhibitor. In some embodiments, ERG is ERG1.

In some embodiments, the expression of ETV2 in the non-vascular cells is transient, and the expression of FLI1 and ERG is constitutive. In some embodiments, ETV2 is transiently expressed the non-vascular cells for 13-15 days. In some embodiments, the non-vascular cells are transduced with vectors comprising nucleic acids encoding transcription factors ETV2, FLI1 and ERG to achieve expression of the transcription factors. In some embodiments, mRNAs encoding the transcription factors ETV2, FLI1 and ERG are delivered into non-vascular cells to achieve expression of the transcription factors.

In some embodiments, the TGFβ signaling inhibitor is present in the cell culture for 20-24 days. In some embodiments, the TGFβ signaling inhibitor is an inhibitor specific for the type I TGFβ receptors. In some embodiments, the TGFβ signaling inhibitor is a polypeptide comprising a soluble form of a type I TGFβ receptor, an antibody directed to a type I TGFβ receptor or ligand, or a small molecule compound. In some embodiments, the TGFβ signaling inhibitor is a small molecule compound selected from SB-431542, A 83-01, D 4476, LY 364947, SB 525334, SD 208, and SJN 2511. In a specific embodiment, the TGFβ signaling inhibitor is SB-431542.

In some specific embodiments, the non-vascular cells are cultured for at least 21 days with the expression of ETV2 in the non-vascular cells for the first 13-15 days, the presence of the TGFβ signaling inhibitor for the first 20-21 days, and constitutive expression of FLI1 and ERG.

In some embodiments, the non-vascular cells are cultured for a total duration of at least 28 days. In some embodiments, the non-vascular cells are cultured for a total duration of at least 42 days.

In some embodiments, the non-vascular cells are selected from the group consisting of Amniotic Cells (“ACs”), Embryonic Stem (“ES”) cells, induced pluripotent stem cell (iPS cells), mesenchymal stem cells (MSC), myocardial stem cells, myocardial cells, fibroblasts, myoblasts, chondrocytes, hepatocytes, blood cells, epithelial cells and nerve cells. In some embodiments, the rECs are derived from amniotic cells.

In another aspect, this disclosure provides a substantially pure population of non-vascular cell-derived ECs, wherein the ECs are characterized by expression of surface markers, VE-cadherin, CD31 and VEGFR2, wherein the ECs comprise an exogenously introduced nucleic acid encoding Sox17.

In some embodiments, this disclosure provides a composition comprising a substantially pure population of non-vascular cell-derived ECs disclosed herein and at least one pharmaceutically acceptable carrier or diluents.

In some embodiments, this disclosure provides a method for repairing injured tissue in a human subject, comprising administering to the subject a composition comprising a substantially pure population of non-vascular cell-derived ECs disclosed herein to promote vascularization in the tissue.

In some embodiments, this disclosure provides a method for treating a tumor in a human subject, comprising administering to the subject a composition comprising a substantially pure population of non-vascular cell-derived ECs disclosed herein, wherein the ECs are engineered to deliver an anti-tumor agent, and upon administration, said ECs form vessels into said tumor.

### BRIEF DESCRIPTION OF THE DRAWINGS

FIGS. 1A-1M. Non-vascular mouse amniotic cells can be converted to EC-like cells. (A) Schematic of conversion of adult fibroblasts, embryonic fibroblasts and mid-gestation mouse amniotic cells. (B) Time course of surface expression of VEcad and CD31 on control and Ets transcription factor—infected amniotic cells analyzed flow cytometry (n=6). (C), Surface expression of VEcad and CD31 on Ets transcription factor—infected MACs after day 28 and on cultured Akt-LECs as detected by fluorescent microscopy. Scale bars=100 μM (D) Flow cytometry indicating surface expression of EC and pluripotency markers on amniotic cells (n=5). (E) Amniotic cells were precultured and then depleted of the VEcad⁺ or CD31⁺ cells and conversion efficiency was assessed by surface expression of VEcad and CD31 28 days after initiation of conversion conditions (n=4). (F) Quantitative PCR (QPCR) of pluripotency factors transcript levels in amniotic cells compared to embryonic stem cells (n=5). (G) QPCR detecting transcripts of EC markers expressed in amniotic cells, RACVECs, and Akt-LECs (n=7). (H) Surface expression of EC markers on amniotic fluid cells, RACVECs, and Akt-LECs assessed by flow cytometry (n=5). (I) Hierarchical clustering based on whole-transcriptome analyses of MACs, RACVECs, directly isolated liver ECs (LSECs), Akt-transduced liver ECs, Akt-transduced LECs. (J) An average Akt-LEC sample was calculated and 1-Pearson correlations between expression profiles were calculated to represent the proximity of each sample to the average Akt-LEC sample. (K) GO term analysis using the set of genes in which FPKM values were upregulated by more than log2, P<0.05, for RACVECs versus amniotic cells. Genes upregulated in converted cells were enriched for EC and EC-related terms. (L) GO term analysis using the set of genes in which FPKM values were downregulated by more than log2, P<0.05, for RACVECs versus MACs. For all panels, bar heights indicate means, error bars indicate standard deviations among biological replicates, asterisks indicate p<0.05 and double asterisks indicate p<0.01 two-sided t-tests, assuming normal distribution. (M) Cells were grown in conversion conditions for 7 days and then the CD31-positive and negative fractions were isolated by FACS. Surface expression of VEcad and CD31 were subsequently analyzed by flow cytometry at day 35 (n=3).

FIG. 2A-2I. Constitutive Akt-signaling endows RACVECs with EC functions. (A) In vitro network quantification. Branching was scored by dividing the number of beads connected to another bead by an EC tube over the total number of beads in a given field (n=3). (B) EC branching, detected by fluorescent microscopy, between beads for RACVECs, Akt-RACVECs, and Akt-LECs. Scale bars=100 μM. (C) In vivo tubulogenesis of MACs, Akt-MACs. RACVECs, Akt-RACVECs, and Akt-LECs. Seven days after implantation, mice sacrificed and plugs were dissected and photographed with a millimeter ruler. (D) Dissected matrigel plugs from mice that were retro-orbitally injected with fluorescently labeled anti-VEcad antibody and plugs analyzed by fluorescent microscopy. White arrows point at unincorporated GFP⁺ cells while orange arrows point at engrafted GFP⁺intravital VEcad⁺ cells. Scale bars=50 μM. (E) Matrigel plugs were dissociated. Resulting cell preparations were analyzed by flow cytometry. The % intravital VEcad⁺ on the y-axis represents the percent of labeled cells recovered from the plug that were also stained by intravital fluorescently-labeled anti-VEcad antibody (n=4). (F) Mice were hepatectomized and injected intrasplenically with fluorescently-labeled RACVECs or Akt-RACVECs. 14 days later they were retro-orbitally injected with fluorescently-labeled anti-VEcad antibody and analyzed by fluorescent microscopy. Green arrows point at GFP⁺ cells, orange arrows point at engrafted GFP⁺ intravital VEcad⁺ cells. Scale bars=50 μM. Three independently isolated Akt-RACVECs were tested in three experiments and for all isolates, engraftment was observed. (G) Hierarchical clustering based on whole-transcriptome analyses. (H) GO term analysis using the set of genes in which FPKM values were increased by more than log2, P<0.05, for Akt-RACVECs versus RACVECs. (I) Heatmap with genes from the vessel and branching morphogenesis GO categories shown in FIG. 2h. Colors reflect Z-scores of individual isolates with blue representing 0 and red representing the maximum FPKM value, 1, for a given transcript. For all panels, bar heights indicate means, error bars indicate standard deviations among biological replicates, asterisks indicate p<0.05 and double asterisks indicate p<0.01 in two-sided t-tests, assuming normal distribution.

FIG. 3A-3I. Akt-signaling steers the genomic targeting of Fli1 towards endothelial genes. (A) Venn diagram indicating overlapping DBRs. (B) The log-odds ratio of the overlap between the indicated refGene elements (top graph) and the genomic locations of the indicated Fli1 DBRs is shown. Grey is a control set of random genomic locations with the same median bp length as the Fli1 bound regions. The lower graph indicates the overlap between the indicated regulatory regions derived from ENCODE chromatin states for HUVECs and the genomic locations of the Fli1-bound regions for each cell type. (C) Heatmaps of selected loci of EC genes. The mean binding signal for biological replicates is shown in blue scale. Gene structures are represented with white bars (exons) and blue (introns). (D) Dendogram of Fli1-binding in all samples depicts similarity by k-means distance/clustering based on normalized Fli1-binding logFC. (E) Venn diagram indicating overlapping and unique regions. Purple represents sites in Akt-RACVEC set, and the darker blue represents RACVEC sites that are downmodulated compared to Akt-RACVEC, the light blue represents sites that are absent in all other cell types but RACVEC, and the slice of orange represents sites upmodulated in Akt-RACVECs. (F) Overlap between murine elements homologous to chromatin states ENCODE defined as active promoter and the indicated Fli1 binding site sets. The probabilities are indicated on the y-axis and the cell types whose regulatory regions were analyzed are indicated on the x-axis. (G) Overlap between murine genomic elements homologous to Fli1 bound regions in HUVECs (Patel, M. et al., Genome Res, 22, 259-270, (2012)) and the genomic locations of Fli1 bound regions. (H) Enrichment of motifs representative of specific families is shown for the indicated datasets. Inverse log E-values are indicated on the y-axis (higher is more enriched). (I) QPCR detecting transcripts of members of the Sox family of transcription factors among MAC, Akt-MAC, RACVEC, Akt-RACVEC, and Akt-LEC (n=5). Bar heights indicate means, error bars indicate standard deviations among biological replicates tested in experiments performed at different times, asterisks indicate p<0.05 according to two-sided t-tests, assuming normal distribution.

FIG. 4A-4O. Sox17 enhances conversion and endows RACVECs with EC functions. (A) In vitro network quantification. (B) EC connections between beads with Sox17-RACVECs. Scale bar=100 μM. (C) In vivo tubulogenesis of RACVECs, Sox17-RACVECs, and Akt-LECs. Plugs were dissected and photographed with a millimeter ruler. (D) Matrigel plugs were fixed and sectioned and analyzed by fluorescent microscopy. Orange arrows point at engrafted GFP⁺intravital VEcad⁺ cells. Scale bars=50 μM (n=3). (E) Matrigel plugs were dissociated and analyzed by flow cytometry. The % intravital VEcad⁺ represents the percent of labeled cells recovered from the plug that were stained by intravital anti-VEcad antibody (n=3). (F) Two mice were hepatectomized and injected intrasplenically with GFP-labeled Sox17-RACVECs. Liver sections were analyzed by fluorescent microscopy. Orange arrows point at engrafted GFP⁺intravital VEcad⁺ cells. Scale bars=50 μM. (G) Mice that underwent unilateral artery excision and cell transplantation were injected with fluorescently labeled anti-VEcad antibody and sacrificed. Images of mice from day 14. Orange arrows point at engrafted GFP⁺intravital VEcad⁺ cells. Scale bars=200 μM. (H), Thigh muscles were dissociated and analyzed by flow cytometry 14 days after injury and cell injection. The % intravital VEcad⁺ represents the percent of labeled cells recovered from the muscle that were stained by intravital anti-VEcad antibody (n=3). (I) At days 1, 4, 7, 14, and 21 reperfusion was measured as the ratio of intensity measured in the affected limb over the unaffected limb (n=8). Bar heights indicate means, error bars indicate standard deviations among individual male animals, asterisks indicate p<0.05 and double asterisks indicate p<0.01 two-sided t-tests assuming normal distribution. (J) and (M) FPKMs indicating transcript levels of indicated genes (n=3). (K) and (N) GO term analysis using the set of genes in which FPKM values were increased by more than log2, P<0.05, for Sox17-RACVECs versus RACVECs and vice versa. (L) and (O) Heatmaps of representative cell types with selected genes. Colors reflect Z-scores of individual isolates with blue representing 0 and red representing the maximum FPKM value, 1.

### DETAILED DESCRIPTION

### Definitions

As used herein, the term “about” refers to an approximately ±10% variation from a given value.

As used herein, the term “engraftability” of endothelial cells refers to the ability of transplanted cells to form new vessels after transplantation in a recipient and integrate into existing vessels of the recipient.

As used herein, the term “functionality” of endothelial cells refers to the ability of transplanted cells to incorporate into existing vessels or to form new vessels after transplantation, and the vessel comprising the transplanted cells can perform the functions of vessels. The functions of vessels include carrying out tasks such as transporting oxygen rich blood to cells in the body, and removing carbon dioxide and cellular wastes away from the cells.

As used herein, the term “vascular cells” refers to cells involved in angiogenesis such as cells constituting blood vessels and blood, progenitor cells being able to be differentiated into the aforementioned cells, and somatic stem cells. As used herein, the term “non-vascular cells” refers to cells other than the vascular cells. Examples of non-vascular cells include Amniotic Cells (“ACs”), Embryonic Stem (“ES”) cells, Induced pluripotent stem cell (iPS cells), mesenchymal stem cells (MSC), myocardial stem cells, myocardial cells, fibroblasts, myoblasts, chondrocytes, hepatocytes, blood cells, epithelial cells or nerve cells.

### General Description

Disclosed herein are methods of generating functional and durable endothelial cells with improved functionality and engraftability.

In some embodiments, the method comprises enforced expression of the transcription factor Sox17 in reprogramming-derived endothelial cells (rECs). In some embodiments, rECs are produced by enforced expression of ETS family transcription factors and simultaneous inhibition of the TGFβ signaling pathway. The Sox17-expressing rECs (Sox17-rECs) are superior over rECs that do not express Sox17 by having improved function and engraftability.

In some embodiments, rECs are derived from amniotic cells and are called RACVECs.

Enforced Expression of Sox17 in rECs (Sox17-rECs)

The inventors of this disclosure discovered that exogenously expressing the transcription factor Sox17 in reprogramming-derived endothelial cells (rECs) improves the functionality and engraftability of the rECs. In this disclosure, rECs that exogenously express Sox17 are referred to as “Sox17-rECs”. Sox17 is not expressed in naïve rECs (including RACVECs), but is found in ECs isolated from adult tissue and obtained by directed differentiation of pluripotent cells.

Sox17 (SRY (Sex Determining Region Y)-Box 17) nucleic acid and protein sequences are available from GenBank and other databases (e.g., GENBANK Accession NO: NM_022454.3, HGNC: 18122, Entrez Gene: 64321, Ensemb1: ENSG00000164736, OMIM: 610928, UniProtKB: Q9H6I2). Sox17 acts as transcription regulator that binds target promoter DNA and bends the DNA. Sox17 binds to the sequences 5-AACAAT-3 or 5-AACAAAG-3. Multiple isoforms of Sox17 have been characterized in mouse and likely exist for humans (Kanai et al., JCB, 1996 133(3) 667-681).

Accordingly, the present approach involves enforced expression of the transcription factor Sox17 in rECs. In some embodiments, rECs are transduced with a vector comprising a nucleic acid encoding Sox17 to achieve expression of Sox17. In some embodiments, an mRNA encoding Sox17 is delivered into rECs to achieve expression of Sox17. In some embodiments, Sox17 is delivered into rECs as polypeptides to achieve expression of Sox17.

In some embodiments, Sox17 is constitutively expressed in rECs. In some embodiments, Sox17 is constitutively expressed from a mammalian expression vector comprising a promoter selected from the list consisting of SV40 early or late promoters, cytomegalovirus (CMV) immediate early promoters, Rous Sarcoma Virus (RSV) early promoters, beta actin promoter, GADPH promoter, metallothionein promoter; cyclic AMP response element promoters (cre), serum response element promoter (sre), phorbol ester promoter (TPA) and response element promoters (tre). In some embodiments, Sox17 is constitutively expressed from a lentivirus vector. In a specific embodiment, the lentivirus vector Sox17 cDNA is cloned into is Lv203 (Genecopeia) lentivirus vector or pCCL-PGK lentivirus vector. In some embodiments, Sox17 could be expressed by modified RNAs that are resistant to intracellular degradation. In some embodiments, Sox17 gene or protein could be packaged in exosomes. Exosomes are cell-derived vesicles that are present in many and perhaps all eukaryotic fluids, including blood, urine, and cultured medium of cell cultures (van der Pol E, Böing et al., Pharmacol. Rev., 64 (3): 676-705; Keller S. et al., Immunol. Lett., 107 (2): 102-8.). A sub-type of exosomes, defined as matrix-bound nanovesicles (MBVs), was reported to be present in extracellular matrix (ECM) bioscaffolds (non-fluid) (Huleihel, Luai et al.; Science Advances. 2 (6): e1600502). The reported diameter of exosomes is between 30 and 100 nm, which is larger than low-density lipoproteins (LDL) but much smaller than, for example, red blood cells. Exosomes are either released from the cell when multivesicular bodies fuse with the plasma membrane or released directly from the plasma membrane (Booth A M. et al., J. Cell Biol., 172 (6): 932-935). Exosomes offer distinct advantages that uniquely position them as highly effective carriers. Exosomes can be used to transport drugs, genes or other therapeutics to cells. Composed of cellular membranes with multiple adhesive proteins on their surface, exosomes are known to specialize in cell—cell communications and provide an exclusive approach for the delivery of various therapeutic agents to target cells (Batrakova E. V. et al.; Journal of Controlled Release. 219: 396-405.).

In some embodiments Sox17 is constitutively expressed in rECs for at least 20 days before Sox17-rECs are transplanted into a patient. In some embodiments, Sox17 is constitutively expressed in rECs for 20 days, 22 days, 24 days, 26 days, 28 days, 30 days, 35 days, 40 days or 42 days before Sox17-rECs are transplanted into a patient.

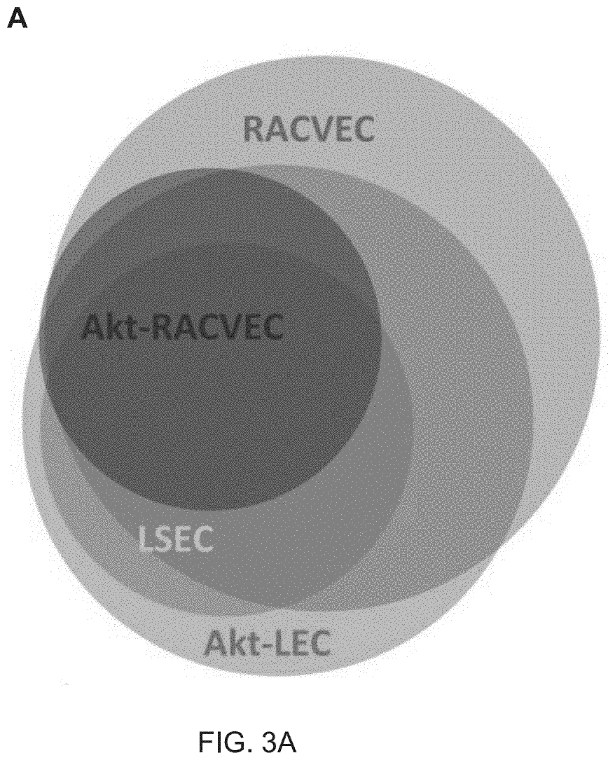

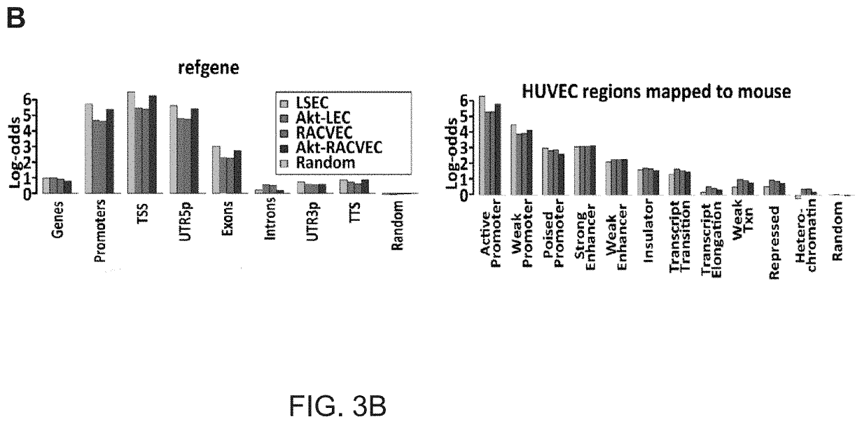

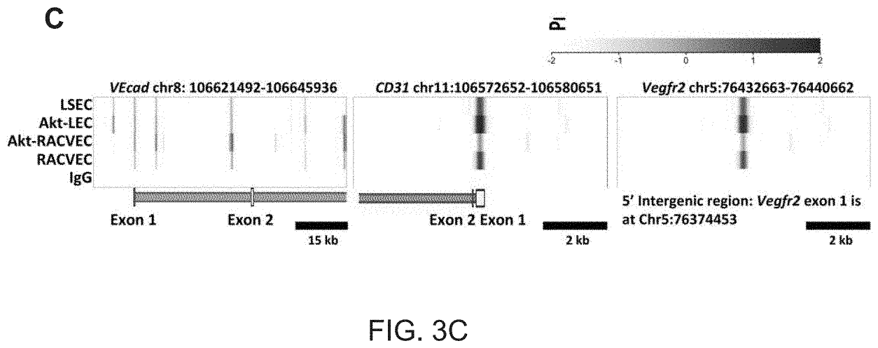

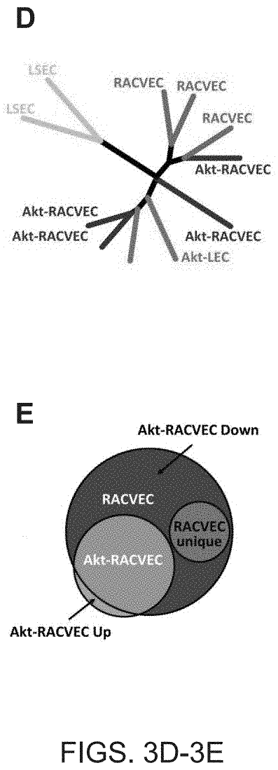

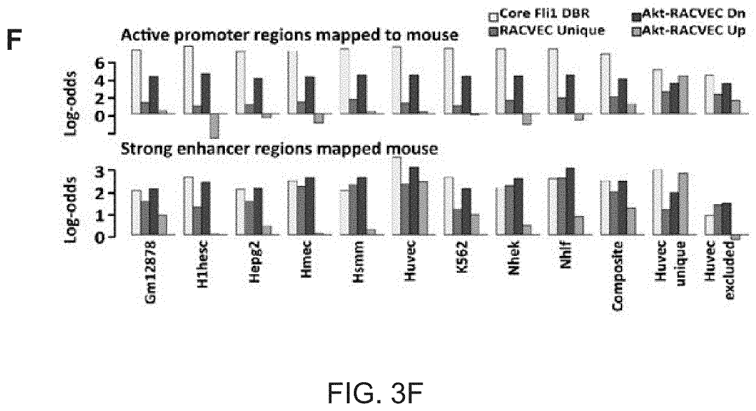

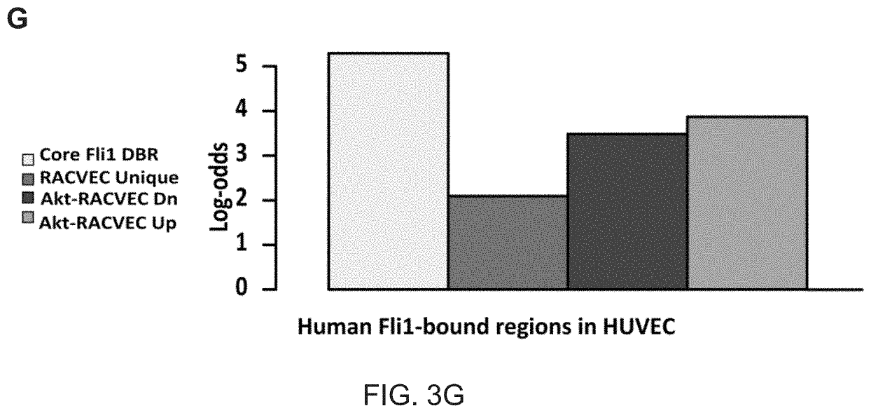

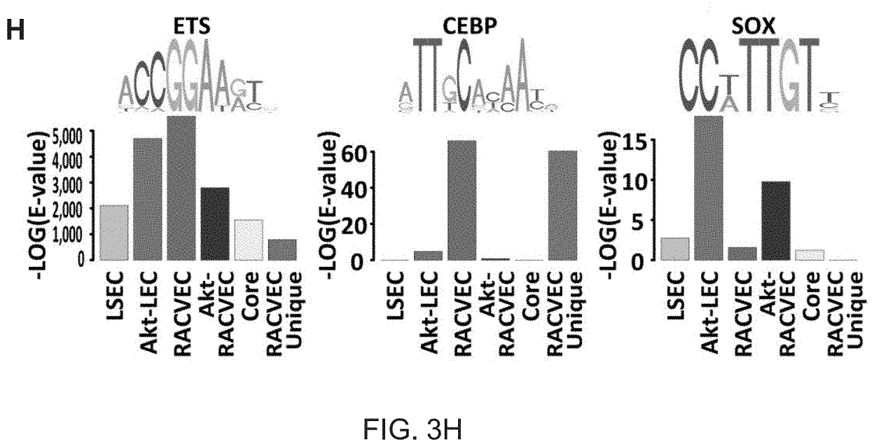

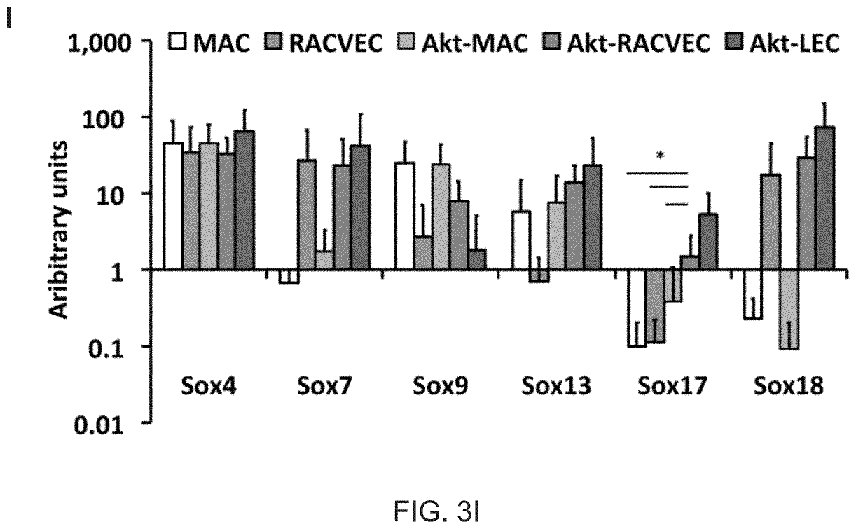

In some embodiments, Sox17-rECs can be used to elucidate the cellular and molecular mechanisms of endothelial engraftment after transplantation. Without limiting the disclosure to a specific theory, since Sox17 is a transcription factor that promotes engraftment, it is reasonable to expect that it achieves this by modifying expression of genes that promote engraftment. Analysis of Sox17-RACVEC gene expression by the inventors indicates that Sox17 enhances expression of angiogenic genes, such as Vegfr2, and cell-adhesion proteins, such as ZO-2 and CD31 (FIG. 3a-d). It could be determined how Sox17 modifies their expression, i.e. directly or indirectly, and their contribution to engraftment could be tested by loss- and gain-of-function studies using in vivo tests of EC function. Engraftment of Sox17-RACVECs could also be subjected to pharmacological-based assays in which matrigel plugs or mouse recipients are exposed to small molecules that might reduce or improve engraftment.

In some embodiments, Sox17-rECs are cultured for a total duration of at least 20 days, 22 days, 24 days, 26 days, 28 days, 30 days, 35 days, 40 days or 42 days before Sox17-rECs are transplanted into a patient.

Thus, this invention also provides a substantially pure population of stable Sox17-rECs. By “substantially pure” it is meant that Sox17-rECs account for at least 75% , 80% , 85% , 90% , 95% , 98% , 99% or greater percentage of the cells in the cell population. By “stable” it is meant that Sox17-rECs can be cultured for extended period of time, e.g., at least 5 passages, at least 10 passages, at least 15 passages or longer, without losing the characteristics of Sox17-rECs.

Sox17-rECs display enhanced engraftment and reperfusion in revascularization as compared to rECs that do not express Sox17. Sox17-rEC engraftment is also long-lasting, and vessel-integrated cells can be observed two months after transplantation. Sox17-rECs have substantially the same morphological features as RACVECs under a light microscope. Cell surface markers characteristic of Sox17-rECs include at least VE-cadherin⁺, VEGFR2⁺, and CD31⁺, and also optionally, EC-Selective Adhesion Molecule (ESAM) and Junctional Adhesion Molecule A (JAM-A), all of which are expressed on adult ECs. The transcriptional profile of Sox17-rECs is characterized by downregulation of genes including Mmp3, Grem1, Gas1, and Notch2 genes as compared to rECs that do not express Sox17 and other EC cells (e.g., liver EC or LSEC). The transcriptional profile of Sox17-rECs is also characterized by upregulation genes including of Col18al, CD31, Tjp2 (ZO-2), and Vegfr2 as compared to rECs that do not express Soxl7and other EC cells (e.g., liver EC or LSEC).

The Sox17-rECs can be used directly in therapeutic applications or cryopreserved for future use using conventional cryopreservation methods.

### Pharmaceutical Compositions and Therapeutic Methods

The methods disclosed herein permits reproducible production of large numbers of functional and stable improved Sox17-rECs, which are bankable, and can be HLA-typed, and therefore will be useful for therapeutic vascularization of injured tissues.

Accordingly, in a further aspect, this disclosure provides a composition containing Sox17-rECs. The composition can include one or more pharmaceutically acceptable carriers and diluents. The composition can also include components that facilitate engraftment.

In still a further aspect, this disclosure is directed to therapeutic uses of the endothelial cells provided herein. For example, the instant endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the instant endothelial cells can be further modified to deliver agents to target and treat tumors.

In specific embodiments, this disclosure provides a method of repair or replacement for tissue in need of vascular cells or vascularization. This method involves administering to a human subject in need of such treatment, a composition containing the isolated Sox17-rECs to promote vascularization in such tissue.

The tissue in need of vascular cells or vascularization can be a cardiac tissue, liver tissue, pancreatic tissue, renal tissue, muscle tissue, neural tissue, bone tissue, brain tissue, reproductive tissues, endorcrine tissues, among others, which can be a tissue damaged and characterized by excess cell death, a tissue at risk for damage, or an artificially engineered tissue.

Promoting angiogenesis and restoring tissue-specific angiocrine (paracrine function) of endothelial cells in a tissue can be beneficial to individuals who have or are at risk to develop a condition including an ischemic condition, e.g., myocardial infarction, congestive heart failure, and peripheral vascular obstructive disease, stroke, reperfusion injury, limb ischemia; neuropathy (e.g., peripheral neuropathy, or diabetic neuropathy), organ failure (e.g., liver failure, kidney failure, and the like), diabetes, rheumatoid arthritis, and osteoporosis.

The Sox17-rECs of this invention or a composition containing such cells can be administered in a manner that results in delivery or migration to or near the tissue in need of repair or vascularization. In some embodiments, the cells are systemically administered and circulate to the tissue in need thereof; or alternatively, locally administered, e.g., delivered directly (by injection, implantation or any suitable means) into the tissue or nearby tissue which is in need of these cells. In other embodiments, the cells are integrated into an artificially engineered tissue prior to implantation.

In another embodiment, this disclosure provides a method of targeting certain agents to tumors in a subject by administering to the subject the endothelial cells that have been engineered for delivery of such agents. Because tumors frequently stimulate the in-growth of new blood vessels into the tumor (stimulate tumor angiogenesis), endothelial cells delivered to a subject can contribute to the new tumor vasculature. Thus, the cells can be used to deliver agents directly to a tumor site. Examples of agents that can be targeted to tumors using endothelial cells include, but are not limited to, cytotoxic drugs, other toxins, radionuclides, and gene expression products. For example, endothelial cells can be engineered such that they also express a protein having anti-tumor activity, or such that they secrete, release, or are coated with a toxic agent such as a chemotherapeutic agent or radionuclide. For example, radionuclide drugs or chemotherapeutic drugs can be conjugated to an antibody that binds to the surface of the endothelial cells and thereby used to deliver the radionuclides or chemotherapeutic drugs to a tumor.

### Expressing Transcription Factors in Cells

To achieve expression of a transcription factor in non-vascular cells or reprogramming-derived endothelial cells (rECs), a nucleic acid encoding the transcription factor can be delivered into the cells using various vectors, which include integrative vectors which integrate into host cells genome by either random integration or targeted integration via homologous recombination, and episomal vectors that are maintained extra-chromosomally. In addition to delivery by vectors, a nucleic acid encoding a transcription factor can also be delivered to cells in the form of mRNAs, as described in Yamamoto et al. (Eur. J. Phar. Biophar 71: 484-89 (2009). Alternatively, Sox17 could also transiently be delivered by modified RNAs. In a specific embodiment, the modified RNAs are resistant to intracellular degradation.

Examples of delivery vectors include but are not limited to, plasmids, cosmids, viruses (bacteriophage, animal viruses, and plant viruses), and artificial chromosomes (e.g., YACs). Viral vectors include e.g., retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, and Rous sarcoma virus vectors.

In specific embodiments, a nucleic acid encoding a desirable transcription factor is delivered via a lentiviral vector. Lentiviral vectors are well known in the art (see, for example, U.S. Pat. Nos. 6,013,516 and 5,994,136) and can provide strong and sustained expression for several months.

Those skilled in the art can clone a nucleic acid encoding a transcription factor into a suitable vector using available molecular biology techniques. The vectors can include additional sequences appropriate, such as a 5′ regulatory sequence (e.g., a promoter, an enhancer, or a combination thereof), a 3′ transcription termination sequence, one or more origins of replication, or a selection marker. The promoter in the vector can be one naturally associated with the transcription factor, and can also be a heterologous promoter that achieves effective expression of the transcription factor in non-vascular cells. Examples of promoters suitable for use herein include, but not limited to, SV40 early or late promoters, cytomegalovirus (CMV) immediate early promoters, Rous Sarcoma Virus (RSV) early promoters, beta actin promoter, GADPH promoter, metallothionein promoter; cyclic AMP response element promoters (cre), serum response element promoter (sre), phorbol ester promoter (TPA) and response element promoters (tre).

Introduction of a nucleic acid encoding a transcription factor, such as DNA or RNA, in the form of a vector or virus, into ACs, can be achieved using various suitable methods. Such methods include, but are not limited to, liposome-mediate transfection, electroporation, calcium phosphate precipitation, DEAE-Detxan followed by polyethylene glycol, sonication loading, microprojectile bombardment, and any combination of any of the above well known techniques.

In addition to nucleic acid delivery, in some embodiments, transcription factors are provided to non-vascular cells via direct delivery of polypeptides, also referred to as protein transduction. In protein transduction, a desirable transcription factor is fused to a protein transduction domain (or “PTD”) which can cross a cell membrane and delivers the fusion protein into ACs. Examples of PTDs are described in Ho et al., Cancer Research 61(2):474-7 (2001) (HIV Tat), WO03/059940 (human SIM-2), WO03/059941 (Mph), Rothbard et al. (Nature Med. 6(11): 1253-7 (2000), among others. Similarly, the gene or protein of Sox17 can be delivered by packaging into exosomes, nanoparticles, liposomes or membraneless droplets.

Reprogramming-Derived Endothelial Cells (rECs)

The term “ reprogramming-derived endothelial cells” or “rECs” is used to refer to vascular endothelial cells generated from non-vascular cells using the reprogramming scheme disclosed herein.

In a specific embodiment, the non-vascular cells are amniotic cells. rECs derived from amniotic cells are called “amniotic cells reprogrammed into vascular endothelial cells” or “rAC-VECs” (see U.S. Pat. No: 9,637,723). rAC-VECs are distinguished from the adult vascular endothelial cells isolated from a mammalian subject, such as human umbilical vein endothelial cells (HUVECs) and adult liver sinusoidal ECs (“LSECs”), despite the fact that rAC-VECs and adult ECs share similar morphological features, cell surface phenotypes, and transcription profiles. For example, rAC-VECs, like HUVECs, are about 10 μm in length, and of a “fried-egg” or cobblestone shape. Cell surface markers characteristic of rAC⁻VECs include at least VE-cadherin⁺, VEGFR2⁺, and CD31⁺, and also optionally, EC-Selective Adhesion Molecule (ESAM) and Junctional Adhesion Molecule A (JAM-A), all of which are expressed on adult ECs. The transcriptional profile of rAC-VECs is characterized by expression of VE-cadherin, VEGFR2, CD31⁺, expression of angiocrine factors including BMPs, Notch-ligands, IGFs, CSFs, Kit-ligand, semaphorins, and EGFL7; lack of expression of non-EC genes such as smooth muscle actin, musclin, calponin-1, and natriuretic peptide B; and negative for hemapoietic markers including CD45, CD15, Pu.1, TPO-receptor, Flt3 receptor or Lhx2.

Emergence of rAC-VECs in the ACs culture can be determined based on growth characteristics, morphological features, cell surface phenotypes, transcription profiles, or a combination of any of these characteristics. It has been shown that transduction of ACs with ETV2/FLI1/ERG1 not only resulted in complete induction of a vascular signature, it also turned off non-vascular programs in ACs (see U.S. Pat. No: 9,637,723, which is incorporated by reference in its entirety). rAC-VECs are highly proliferative and stable, capable of undoing 6×10⁴-fold expansion in 50 days, while maintaining their full angiogenic repertoire. If desirable, rAC-VECs can be isolated from the cell culture using antibodies specific for EC surface markers, such as VE-cadherin, CD31 or VEGFR2, attached to magnetic beads or fluorophores for use in Magnetic or Fluorescence Activated Cell Sorting (MACS or FACS).

### Non-Vascular Cells

Non-vascular cells of the present disclosure include Amniotic Cells (“ACs”), Embryonic Stem (“ES”) cells, Induced pluripotent stem cell (iPS cells), mesenchymal stem cells (MSC), myocardial stem cells, myocardial cells, fibroblasts, myoblasts, chondrocytes, hepatocytes, blood cells, epithelial cells or nerve cells.

In some embodiments, the non-vascular cells used for the methods of the present disclosure comprise amniotic cells. As used herein, the term “amniotic cells” (or “ACs”) refers to cells extracted from amniotic fluid, and hence also referred to herein as “amniotic fluid cells”.

Although ACs are preferably isolated from human amniotic fluid, they can be isolated from amniotic fluid of other mammalian species as well. Examples of mammalian species suitable for use to collect amniotic fluid include but are not limited humans, primates, dogs, cats, goats, elephants, cattle, horses, pigs, mice, rabbits, and the like. The endothelial cells developed from ACs of a given species can be applied therapeutically to a subject of the same species.

For purposes of this disclosure, ACs can be extracted from amniotic fluid collected from a pregnant female during any stage of gestation. In some embodiments, amniotic fluid is collected during mid-gestation. In certain embodiments, amniotic fluid is collected during week 10-25 of a woman's pregnancy. In other embodiments, amniotic fluid is collected during the second trimester, i.e., week 14-26, of a pregnant woman. In specific embodiments, amniotic fluid is collected during week 16-21 of a woman's pregnancy. In addition, to amniotic cells any adult non-vascular cells (including, but not limited to, epithelial cells, mesenchymal cells, fibroblasts, etc.) can be amenable for reprogramming by ETV2/Fli1/ERG into vascular cells.

ACs or non-vascular cells can be extracted from amniotic fluid by conventional means, for example, centrifugation. The cell pellet can be resuspended in an appropriate medium for immediate use in a reprogramming regimen disclosed herein, or resuspended in a culture medium (e.g., commercially available “Amniotic Media”, exemplified herein below) and cultured for a period of time prior to reprogramming Alternatively, the extracted ACs can be cryopreserved (and e.g., “banked”) for use in the future using conventional techniques. For example, ACs, which are ready for cryopreservation, can be retrieved from culture (e.g., flasks, plates, etc.), e.g., using Accutase (EBioscience #00-4555-56), and then spun down. The cell pellet can then be resuspended in an appropriate media for cryopreservation (e.g., media consisting of 90% FBS (Omega Scientific #FB-11) and 10% DMSO (Cellgro #25-950-COC) and transferred to a cryo-tube. The cells can be stored at −80° C. for at least 3 days (initial freeze), and then transferred to liquid nitrogen (long-term freeze).

ACs or non-vascular cells are typically heterogeneous in terms of the cellular constituents, and include both multipotent cells (e.g., c-Kit⁺ACs) and mature ACs (c-Kit⁻ ACs). Mature ACs include both lineage-committed and non-committed cells. The reprogramming approach disclosed herein is effective in generating rAC-VECs from extracted ACs or non-vascular cells, which include both multipotent and mature cells. The reprogramming approach disclosed herein is also effective in generating rAC-VECs from mature c-Kit⁻ ACs, including from both lineage-committed EpCam⁺Tra1-81⁻c-Kit⁻ epithelioid and EpCam⁻Tral-81⁻c-Kit⁻ non-epithelioid (mesenchymal/fibroblastic) ACs and adult ortholog non-vascular cells. In other words, one can reprogram extracted heterogeneous ACs directly, or reprogram a more mature subpopulation of ACs, although it is not necessary to process extracted ACs in order to isolate a more mature subpopulation for purposes of generating rAC-VECs.

Reprogramming Non-Vascular Cells into Reprogramming-Derived Endothelial Cells (rECs)

As previously described and also disclosed herein, non-vascular cells can be reprogrammed into a proliferative population of stable reprogramming-derived endothelial cells (rECs). In some embodiments, the non-vascular cells are amniotic cells (ACs), and reprogrammed ACs are called rAC-VECs (“amniotic cells reprogrammed into vascular endothelial cells”) or non-vascular adult or fetal cells. The reprogramming involves enforced expression of transcription factors from the ETS family (ETS-TFs) in non-vascular cells in conjunction with suppression of the TGFβ signaling pathway.

### Expression of ETS-TFs in Non-Vascular Cells

In accordance with this disclosure, the ETS-TFs involved in the reprogramming include ETV2 (human ETV2 also known as ER71 or Estrp), FLI1, and ERG. Particular useful isoforms of ERG include ERG1 and ERG2, while other isoforms such as ERG3 and ERG4 may be suitable as well. These ETS-TFs have been described in the art (Lee et al., Cell stem cell, 2: 497-507 (2008); Sumanas et al., Blood, 111: 4500-4510 (2008)); Liu et al., Current Bio. 18: 1234-1240 (2008); McLaughlin et al., Blood, 98: 3332-3339 (2001)), and their nucleic acid and protein sequences are also available from GenBank (ETV2: NCBI Accession No. NM_014209.2, GI: 153791177; ERG1: Accession No. NM_182918.3; GI: 209954798; ERG4: Accession No. NM_001136155.1; GI: 209954807).

It is disclosed herein that ETV2 is central for the induction of an EC fate, whereas ERG and FLI1 promote EC maturity. For example, ETV2 alone can turn on the expression of the vascular markers, VE-cadherin and VEGFR2, but not CD31. In contrast, ERG1 or FLI1 can activate CD31 expression, but not some other key EC markers that are turned on by ETV2.

Accordingly, the present approach of reprogramming of non-vascular cells involves enforced expression of a combination of ETV2, FLI1 and ERG in non-vascular cells. In some embodiments, the reprogramming involves enforced expression of a combination of ETV2, FLI1 and ERG1.

To achieve expression of a transcription factor in non-vascular cells, a nucleic acid encoding the transcription factor can be delivered into non-vascular cells using various vectors, which include integrative vectors, which integrate into host cells genome by either random integration or targeted integration via homologous recombination, and episomal vectors that are maintained extra-chromosomally. In addition to delivery by vectors, a nucleic acid encoding a transcription factor can also be delivered to non-vascular cells in the form of mRNAs, as described in Yamamoto et al. (Eur. J. Phar. Biophar 71: 484-89 (2009). In some embodiments, transcription factors are delivered, for transient expression, by modified RNA, or by exosomes, nanoparticles, liposomes or membraneless droplets.

Examples of delivery vectors include but are not limited to, plasmids, cosmids, viruses (bacteriophage, animal viruses, and plant viruses), and artificial chromosomes (e.g., YACs). Viral vectors include e.g., retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors, adeno-ssociated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, and Rous sarcoma virus vectors.

In specific embodiments, a nucleic acid encoding a desirable transcription factor is delivered via a lentiviral vector. Lentiviral vectors are well known in the art (see, for example, U.S. Pat. Nos. 6,013,516 and 5,994,136) and can provide strong and sustained expression for several months.

Those skilled in the art can clone a nucleic acid encoding a transcription factor into a suitable vector using available molecular biology techniques. The vectors can include additional sequences appropriate, such as a 5′ regulatory sequence (e.g., a promoter, an enhancer, or a combination thereof), a 3′ transcription termination sequence, one or more origins of replication, or a selection marker. The promoter in the vector can be one naturally associated with the transcription factor, and can also be a heterologous promoter that achieves effective expression of the transcription factor in non-vascular cells. Examples of promoters suitable for use herein include, but not limited to, SV40 early or late promoters, cytomegalovirus (CMV) immediate early promoters, Rous Sarcoma Virus (RSV) early promoters, beta actin promoter, GADPH promoter, metallothionein promoter; cyclic AMP response element promoters (cre), serum response element promoter (sre), phorbol ester promoter (TPA) and response element promoters (tre).

Introduction of a nucleic acid encoding a transcription factor, such as DNA or RNA, in the form of a vector or virus, into ACs, can be achieved using various suitable methods. Such methods include, but are not limited to, liposome-mediate transfection, electroporation, calcium phosphate precipitation, DEAE-Dextrannxan followed by polyethylene glycol, sonication loading, microprojectile bombardment, and any combination of any of the above well known techniques.

In addition to nucleic acid delivery, in some embodiments, transcription factors are provided to non-vascular cells via direct delivery of polypeptides, also referred to as protein transduction. In protein transduction, a desirable transcription factor is fused to a protein transduction domain (or “PTD”), which can cross a cell membrane and delivers the fusion protein into ACs. Examples of PTDs are described in Ho et al., Cancer Research 61(2):474-7 (2001) (HIV Tat), WO03/059940 (human SIM-2), WO03/059941 (Mph), Rothbard et al. (Nature Med. 6(11): 1253-7 (2000), among others.

### Controlled Expression of ETV2

It has been discovered by the inventors that controlled expression of ETV2 relative to FLI1 and ERG is important for generating mature and proliferative rECs. For example, in a specific embodiment using ACs as non-vascular cells, clonal analysis revealed that stoichiometric ratios of ETV2 relative to FLI1 and ERG1 are important for generation of mature and proliferative rAC-VECs. In specific examples, both FLI1 and ERG1 are expressed in ideal rAC-VEC clones that express mature EC markers such as CD31, and ETV2 expression appears to be inversely proportional to CD31. Additionally, suppression of ETV2 expression after initial transient ETV2expression actually increases the percentage of mature ECs. Accordingly, the reprogramming approach can be fine-tuned such that the expression of ETV2 is controlled so as to obtain a more homogeneous population of mature ECs.

In some embodiments, the reprogramming of non-vascular cells includes a clone selection step after a period of enforced expression of the combination of ETV2, FLI1 and ERG in non-vascular cells, in order to identify clones that express at least one mature EC marker (e.g., CD31) as a result of proper stoichiometric ratios of ETV2 relative to FLI1 and ERG in the clone. For example, at about day 21 after ACs have been transduced with viral vectors encoding ETV2, FLI1 and ERG1, ACs are screened to identify clones that express not only early EC markers (e.g., VE-cadherin and VEGFR2), but also mature EC markers (e.g., CD31). Although day 21 may be a point in time when rAC-VECs are believed to have achieved maximal maturity and therefore may generate ideal clones with high efficiency, clonal expansion of rAC-VECs that have been in culture for as little as 16 days, or preferably 17 or 18 days or longer, or 19 or 20 days or longer, is also considered to be adequate to generate ideal clones.

In other embodiments, the reprogramming of non-vascular cells involves utilizing vectors and/or 5′ regulatory sequences of different expression profiles (e.g., duration and strengths) to deliver and express ETV2 and FLI1/ERG, respectively, in order to achieve proper stoichiometric ratios of ETV2 relative to FLI1 and ERG in recipient non-vascular cells. For example, lentiviral vectors that direct sustained strong expression can be used for FLI1 and ERG1, and adenoviral vectors that direct relatively transient expression can be used for ETV2. Naked DNAs encoding the transcriptional factors may also be appropriate.

In still other embodiments, the reprogramming of non-vascular cells involves transient expression of ETV2, along with constitutive expression of FLI1 and ERG. In specific embodiments, transient expression of ETV2 refers to expression of ETV2 for about 10 to 18 days, 12-16 days, 13-15 days, or about 14 days. In a specific embodiment wherein the non-vascular cells are ACs, a minimum of 10 days is considered to be sufficient time for ETV2 to specify amniotic cells towards an endothelial cell fate. One can assess VEGFR2 and VE-cadherin protein expression to confirm that rAC-VECs are being generated. Furthermore, as ETV2 is shown herein to negatively regulate CD31 (PECAM) expression in rAC-VECs, one can also assay for CD31 subsequent to the shutdown of ETV2. rAC-VECs positive for all three EC protein markers (VEGFR2, VE-cadherin and CD31) after ETV2 shutdown and removal of TGFβ inhibition are committed rAC-VECs.

Transient expression can be achieved by various means, including but not limited to, the use of inducible or conditional expression system (including inducible promoters), a recombinase system, and nucleic acid agents that antagonizes the production or activity of ETV2 mRNA.

In one exemplary embodiment, transient expression is achieved with the Lenti-X™ Tet-Off inducible expression system, which is commercially available from CLONTECH. Briefly, this system utilizes two lentiviral vectors: a regulator vector that stably expresses the Tet-Off transcriptional activator, and a response vector (pLVX-Tight-Puro) that controls the expression of ETV2 gene. Lentiviral particles are produced from each of the vectors and are used in co-transducing non-vascular cells. Expression of the Tet-OFF transcriptional activator from the regulator vector turns on the transcription of ETV2 from the response vector in die absence of doxycyclin. Subsequent suppression of ETV2 expression is achieved via doxycyclin treatment.

In another exemplary embodiment, transient expression of ETV2 is achieved through the use of a recombinase based system, such as Cre/Lox or FLP/FRT. The FLP protein catalyzes site-specific recombination events, and the FLP gene has been cloned from S. cerevisiae (Cox (1993) Proc. Natl. Acad. Sci. U.S.A. 80:4223-4227, incorporated herein by reference). The recombination site recognized by the FLP protein is referred to as FRT, which contains two inverted 13-base pair (bp) repeats surrounding an asymmetric 8-bp spacer: The FLP protein cleaves the site at the junctions of the repeats and the spacer. The ETV2 coding sequence can be placed in a delivery vector between two FRT sites in direct repeat orientation. After the introduction of such a vector into non-vascular cells and a period of ETV2 expression, supply of the FLP recombinase into the cells can lead to deletion of the DNA sequences between the FRT repeats and one of the FRT repeats, leaving a “scar” in the remaining FRT sequence making it illegible for further recombination by FLP. Similarly, the bacteriophage recombinase Cre catalyzes site-specific recombination between two lox sites, leading to the deletion of the sequence between the recombination sites, and with properly selected lox sequences, creation of a “scar” sequence as well. For more details of Cre recombinase, see, Hamilton et al., J. Mol. Biol. 178:481-486 (1984), Sternberg et al. J. Mol. Biol. 187:197-212 (1986), Sauer et al., Proc. Natl. Acad. Sci. (U.S.A.) 85.:5166-5170 (1988), and Sauer et al., Nucleic Acids Res. 17:147-161 (1989), all of which are incorporated herein by reference.

In other embodiments, transient expression of ETV2 is achieved by utilizing nucleic acid molecules that effectively suppress or silence the production or function of ETV2 mRNA. These include antisense RNA, siRNA, and miRNA (or “microRNA”), all of which can be designed based on the gene sequence of ETV2 and introduced into non-vascular cells.

In another embodiment transient expression of ETV2 or other transcription factors can be achieved by the delivery of modified RNA, or packaging these transcription factors in exosomes, nanoparticles, liposomes or membraneless droplets as well as conjugated to other nanoparticles.

Transient expression of ETV2 can also be achieved with naked DNA encoding ETV2 delivered into the cells.

### Inhibition of TGF β Signaling

The present reprogramming approach includes inhibition of TGFβ signaling in conjunction with enforced expression of ETS-TFs. Inhibition of TGFβ signaling, at least for a short term, functionalizes VEGFR2 signaling and augments specification of non-vascular cells to rECs. By “short term” it is meant a period of at least two weeks since the commencement of reprogramming; in specific embodiments, a period of at least 18-19 days; and in other embodiments, at least 20-21 days, e.g., 20-24 days, or about 21 days. The timing of TGFβ inhibition is easily controlled for by addition of either a broad TGFβ inhibitor molecule or an antibody directed against TGFβ ligands to the culture media. VEGFR2 and VE-cadherin protein expression at the cell surface (measurable by FACS) are the two main cellular features one can examine to identify r-VEC generation. Once r-VEC fate has been established (e.g., at about 21 days), TGFβ inhibition is no longer necessary.

Inhibition of TGFβ signaling can be achieved by adding a TGFβ signaling inhibitor to the cell culture of ACs. TGFβ superfamily signaling is mediated by two classes of receptors, the type I or activin like kinase (ALK) receptors, and type II receptors. Type I receptors include ALK4 (type I receptor for activin or inhibin), ALK5 (type I receptor for TGFβ) and ALK7 (type I receptor for nodal).

In certain embodiments, TGFβ signaling inhibitors used herein are selective inhibitors of type I receptors, i.e., inhibitors having differential (i.e., selectivity) for type I receptors relative to type II receptors. Selectivity can be measured in standard assays as an IC₅₀ ratio of inhibition in each assay. The inhibitor can be a specific inhibitor of one type I receptor (i.e., one of ALK4, ALK5 or ALK7), or an inhibitor that inhibits signaling of several type I receptors (e.g., all of ALK4, ALK5 and ALK7).

In a specific embodiment, the inhibitor inhibits at least ALK5-mediated signaling. ALK5, upon activation, phosphorylates the cytoplasmic proteins smad2 and smad3. The phosphorylated smad proteins translocate into the nucleus and activate certain gene expression. Inhibitors of ALK5-mediated signaling can be compounds that inhibit the kinase activity of ALK5 and block phosphorylation of smad proteins. See, e.g., review by Yingling et al., Nature Reviews (Drug Discovery) 3: 1011-1022 (2004).

The inhibitors can be polypeptides, such as soluble forms of TGFβ receptors (e.g., polypeptides composed of the extracellular segment of a receptor), particularly soluble forms of type I receptors, or antibodies directed to a TGFβ receptor particularly a type I receptor or its ligand, e.g., a monoclonal antibody directed to a TGFβ ligand commercially available from R&D: #MAB 1835.

The inhibitors can be small molecule compounds as well. By “small molecule compounds” it is meant small organic compounds, generally having a molecule weight of less than 1200, 1000 or 800 daltons. Small molecule inhibitors of TGFβ signaling have been well-documented in the art, including pyridyl substituted triarylimidazoles disclosed in U.S. Pat. No. 6,465,493 and US 20030149277 A1, pyridyl substituted imidazoles disclosed in US 20030166633 A1 and US 20040220230 A1, pyridyl substituted triazoles disclosed in US 20040152738 A1, thiazolyl substituted triazoles disclosed in US 20040266842 A1, 2-amino-4-(pyridin-2-yl)-thiazole derivatives disclosed in US 20040063745 A1, 2-pyridyl substituted diarylimidazoles disclosed in US 20040039198 A1, phenyl substituted triazoles disclosed in US 20050014938 A1, benzoxazine and benzoxazinone substituted triazoles in US 20050165011 A1 isoquinoline derivatives disclosed in US 20070072901 A1, thiazolylimidazole derivatives disclosed in US 20070154428 A1, heteroaromatic compounds substituted with at least one 2-pyridyl moiety disclosed in U.S. Pat. No. 7,417,041, as well as those reviewed by Yingling et al., Nature Reviews (Drug Discovery) 3: 1011-1022 (2004), the contents of all of these publications are incorporated herein by reference. Small molecule inhibitors are also available through various commercial sources. For example, compounds listed in the following table are available through Tocris Bioscience (Missouri, USA), and are suitable inhibitors for use in the present methods. Additional small molecule inhibitors are available through EMD4Bisciences (New Jersey, USA).

| Compound | Chemical Name/Function |
|---|---|
| A 83-01 | 3-(6-Methyl-2-pyridinyl)-N-phenyl-4-(4-quinolinyl)-1H- |
| | pyrazole-1-carbothioamide |
| | (Selective inhibitor of ALK5, ALK4 and ALK7) |
| D 4476 | 4-[4-(2,3-Dihydro-1,4-benzodioxin-6-yl)-5-(2-pyridinyl)- |
| | 1H-imidazol-2-yl]benzamide |
| | (Selective CK1 inhibitor. Also inhibits ALK5) |
| LY 364947 | 4-[3-(2-Pyridinyl)-1H-pyrazol-4-yl]-quinoline |
| | (Selective inhibitor of ALK5) |
| SB 431542 | 4-[4-(1,3-benzodioxol-5-yl)-5-(2-pyridinyl)-1H-imidazol-2- |
| | yl]benzamide |
| | (selective inhibitor of ALK5, ALK4 and ALK7) |
| SB 525334 | 6-[2-(1,1-Dimethylethyl)-5-(6-methyl-2-pyridinyl)-1H- |
| | imidazol-4-yl]quinoxaline |
| | (Selective inhibitor of ALK5) |
| SD 208 | 2-(5-Chloro-2-fluorophenyl)-4-[(4-pyridyl)amino]pteridine |
| | (Potent ATP-competitive ALK5) |
| SJN 2511 | 2-(3-(6-Methylpyridine-2-yl)-1H-pyrazol-4-yl)-1,5- |
| | naphthyridine |
| | (Selective inhibitor of ALK5) |

In one embodiment, the compound, SB-431542, is used as a TGFβ signaling inhibitor. This compound is added to the culture media at a concentration ranging from about 1 μM to about 15 μM, or about 2 μM to about 10 μM. In a specific embodiment, this compound is added to the media at about 5 μM. Appropriate concentrations for other small molecule inhibitors may depend on the structure or functional mechanism of a particular inhibitor and may be in the micromolar range, which can be determined by those skilled in the art (e.g., based on IC₅₀ values determined in appropriate in vitro assays).

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1

### Materials and Methods

### Cell Culture

Mid-gestation amniotic cells were isolated from pregnant females that were wild type or tdTomato reporter mice in C57BL/6 background, timed according to date (E0.5) of the observed vaginal plug. Amniotic sacs containing embryos aged E11.5-E13.5 were removed from the maternal uterus and placed in saline (PBS). The yolk sac and amnion were gently disrupted, exposing the embryo and releasing the amniotic fluid. The amniotic fluid was washed with PBS and cultured in AmnioMAX (Invitrogen #12001-027), AmnioMAX supplement (Invitrogen #12556-023), and 1× Pen/Strep (Invitrogen #15240-062). After 5 days, the unattached cells were removed by changing media. The cells were cultured for a total of 14 days at 37° in 5% CO₂ before conversion was initiated. Typically, one litter, containing 5-10 embryos, would yield 0.5-1×10⁶ amniotic cells.

MEFs were isolated from E13.5 C57BL/6 embryos. Fetal livers and heads were removed and the remaining tissue was minced with a sterile razor blade. Preparations were incubated with 0.05% trypsin/EDTA (LDP #25-052-CI) for 30 min at 37° in 5% CO₂, further disrupted by pipetting up and down, washed, and then plated in DMEM (LDP #15-013-CM) containing 10% FBS, 1× Pen/Strep (Invitrogen #15240-062). MEFs were pre-cultured for 14 days.

MAFs were isolated from C57BL/6 adult tail-tip and ear tissue, which were minced with a sterile razor blade and incubated with 0.05% trypsin/EDTA (LDP #25-052-CI) for 30 min at 37° in 5% CO₂, then further disrupted by pipetting up and down. Trypsin was quenched by adding equal volume of FBS and the mixture was diluted in DMEM (LDP #15-013-CM) containing 10% FBS, 1× Pen/Strep (Invitrogen #15240-062). MAFs were pre-cultured for 14-days. We considered a biological replicate as an independently isolated culture of MACs or MEFs, which consisted of mixed gendered embryos, and TEFs, which consisted of fibroblasts taken from 1 adult female.

Cells undergoing conversion were grown in EC media, which was composed of 1:1 low glucose DMEM:F12 (LDP #10-013-CV, LDP #10-080-CV), 20% FBS, 1× Pen/Strep (Invitrogen #15240-062), 1× non-essential amino acids (LDP #25-060-CI), 10 mM HEPES (Invitrogen #15630-080), 100 μg mL⁻¹ heparin (Sigma-Aldrich H3149), 50 μg mL-1 endothelial mitogen (Alfa Aeser #J65416), and 5 μM SB431542 (R&D #1614) on tissue plastic coated with fibronectin (Sigma #F1141). During the 28-day conversion process, EC media was supplemented with 20 ng mL-1 mouse VEGF-A (Peprotech #450-32).

For Akt-LEC or Akt-Liver ECs, lung or liver ECs were isolated by magnetic cell sorting, using sheep anti-rat IgG Dynabeads (Life Technologies) pre-captured with an anti-CD31 antibody. Cells were plated and allowed to grow for one day in EC media, then infected with a lentivirus encoding constitutively active myrAkt1 (Akt). 1-3 weeks later, cells were re-purified by FACS using anti-VEcad and anti-CD31 antibodies. Directly purified Liver ECs were isolated by FACS after mice were injected intravitally with fluorescently labeled anti-VEcad and Isolectin 20. For ChIP experiments with LSECs, approximately 6 adult mice, and 1.5×10⁷ of purified cells were used per isolate.

### Lentiviral Vectors and Transduction

Mouse Etv2, Erg, Fli1, and Akt cDNAs were cloned into the pCCL-PGK lentivirus vector. Mouse Sox17 cDNA was cloned into Lv203 (Genecopeia) lentivirus vector. Viruses were produced in 293T cells, concentrated with Lenti-X concentrator (Clontech #631232) and titered using the Lenti-X p24 Rapid Titer kit (Clontech #632200). Cells were infected using MOI 10. Sox17-RACVECs were produced by infecting RACVECs with lentivirus bearing Sox17 and performing puromycin selection.

### Antibodies

Anti-mouse Etv2 (Abcam, EPR5229(2)) (Western blotting 1:100)
Anti-mouse Erg (Abcam, 9FY) (Western blotting 1:100)
Anti-mouse Fli1 (Abcam, ab15289) (Western blotting 1:100)
Anti-mouse CD31 (Biolegend clone 390) (Flow cytometry 1:2000)
Anti-mouse VE-Cadherin (Biolegend clone BV13) (Flow cytometry 1:500)
Anti-mouse Vegfr2 (DC101) (Flow cytometry 1:500)
Anti-mouse CD62e (BD Biosceinces, clone 10E9.6) (Flow cytometry 1:500)
Anti-mouse Itgb1 (BD Biosciences, clone 18/CD29) (Flow cytometry 1:500)
Anti-mouse Meca32 (BD Biosciences, clone MECA-32) (Flow cytometry 1:500)
Anti-mouse Tie2 (Biolegend, clone Tek4) (Flow cytometry 1:500)
Anti-mouse ECadherin (BD Biosciences, Clone 36/E-Cadherin) (Flow cytometry 1:500)
Anti-mouse CD34 (BD Biosciences, Clone RAM34) (Flow cytometry 1:500)
Anti-mouse Vcam (BD Biosciences, Clone 429 MVCAM.A) (Flow cytometry 1:500)
Anti-mouse Sca1 (Biolegend, clone D7) (Flow cytometry 1:500)
Anti-mouse CD41 (Biolegend, clone MWReg30) (Flow cytometry 1:500)
Anti-mouse CD24 (BD Biosciences clone M1/69) (Flow cytometry 1:500)
Anti-mouse VE-Cadherin (R+D #AF1002) (Immunofluorescence 1:100)
Anti-mouse CD31 (Biocare, Clone Mec13.3) (Immunofluorescence 1:100)

### RNA Analysis

RNA was prepared using RNAeasy Mini kit (Qiagen #74106) and 1μg was converted to cDNA using qScript cDNA SuperMix (Quanta #95048-100). Relative transcript levels were determined by quantitative PCR (QPCR), performed on a 7500 Fast Real Time PCR System (Applied Biosystems) using SYBR Green PCR Master Mix (Applied Biosystems). No RT or template control and inspection of dissociation curves verified amplifications. Arbitrary units were determined by normalizing to GAPDH levels. Primer sequences usd in qPCR reactions were as follows:

Mouse Etv2 F
(SEQ ID NO: 1)
5′-AACAAGCATCCATGGACCT-3′
Mouse Etv2 F
(SEQ ID NO: 2)
5′-CTCTGGGAACCCTTTCCAG-3′
Mouse Erg F
(SEQ ID NO: 3)
5′-ACCTCACCCCTCAGTCCAAA-3′
Mouse Erg R
(SEQ ID NO: 4)
5′-TGGTCGGTCCCAGGATCTG-3′
Mouse Fli1 F
(SEQ ID NO: 5)
5′-ATGGACGGGACTATTAAGGAGG-3′
Mouse Fli1 R
(SEQ ID NO: 6)
5′-GAAGCAGTCATATCTGCCTTGG-3′
Mouse VEcad F
(SEQ ID NO: 7)
5′-CACTGCTTTGGGAGCCTTC-3′
Mouse VEcad R
(SEQ ID NO: 8)
5′-GGGCAGCGATTCATTTTTCT-3′
Mouse CD31 F
(SEQ ID NO: 9)
5′-CTGCCAGTCCGAAAATGGAAC-3′
Mouse CD31 R
(SEQ ID NO: 10)
5′-CTTCATCCACCGGGGCTATC-3′
Mouse Vegfr2 F
(SEQ ID NO: 11)
5′-TTTGGCAAATACAACCCTTCAGA-3′
Mouse Vegfr2 R
(SEQ ID NO: 12)
5′-GCAGAAGATACTGTCACCACC-3′
Mouse CD62e F
(SEQ ID NO: 13)
5′-ATGCCTCGCGCTTTCTCTC-3′
Mouse CD62e R
(SEQ ID NO: 14)
5′GTAGTCCCGCTGACAGTA-3′
Mouse Tie2 F
(SEQ ID NO: 15)
5′-ATGTGGAAGTCGAGAGGCGAT-3′
Mouse Tie2 R
(SEQ ID NO: 16)
5′-CGAATAGCCATCCACTATTGTCC-3′
Mouse Sma F
(SEQ ID NO: 17)
5′-GTCCCAGACATCAGGGAGTAA-3′
Mouse Sma R
(SEQ ID NO: 18)
5′-TCGGATACTTCAGCGTCAGGA-3′
Mouse Gapdh F
(SEQ ID NO: 19)
5′-AAATGGTGAAGGTCGGTGTGAACG-3′
Mouse Gapdh R
(SEQ ID NO: 20)
5′-GGTCAATGAAGGGGTCGTTGATGG-3′
Mouse Sox4 F
(SEQ ID NO: 21)
5′-GACAGCGACAAGATTCCGTTC-3′
Mouse Sox4 R
(SEQ ID NO: 22)
5′-GGTGCCCGACTTCACCTTC-3′
Mouse Sox7 F
(SEQ ID NO: 23)
5′-ATGCTGGGAAAGTCATGGAAG-3′
Mouse Sox7 R
(SEQ ID NO: 24)
5′-CGTGTTCTGGTCACGAGAGA-3′
Mouse Sox9 F
(SEQ ID NO: 25)
5′-AGTACCCGCATCTGCACAAC-3′
Mouse Sox9 R
(SEQ ID NO: 26)
5′-ACGAAGGGTCTCTTCTCGCT-3′
Mouse Sox17 F
(SEQ ID NO: 27)
5′-GATGCGGGATACGCCAGTG-3′
Mouse Sox17 R
(SEQ ID NO: 28)
5′-CCACCTCGCCTTTCACCTTTA-3′
Mouse Sox18 F′
(SEQ ID NO: 29)
5′-CCTGTCACCAACGTCTCGC-3′
Mouse Sox18 R
(SEQ ID NO: 30)
5′-GCAACTCGTCGGCAGTTTG-3′

RNA was prepared similarly for RNA Sequencing and the quality was checked on an Agilent Technologies 2100 Bioanalyzer. Libraries were prepared using the TruSeq RNA sample Preparation Kit (Illumina #Rs-122-2001) and sequenced as 2×51 bp reads at the Weill Cornell Genomics Core Facility with the Illumina HiSeq2000 sequencer using paired-end module. After quality control using the Illumina pipeline, reads were mapped using Tophat with default parameters and mouse genome build mmp9 (Trapnell, C. et al. Bioinformatics, 25, 1105-1111, (2009)). Cufflinks with upper-quartile normalization and sequence-specific bias correction was used to generate Fragments per kilobase of transcript per million fragments (FPKM) values (Trapnell, C. et al. Nature Biotechnology 28, 511-515, (2010)).

Hierarchical clustering and principal component analysis were performed in R using log2 transformed FPKM values with distances calculated by subtracting the Pearson correlation value from 1. Those values were also used to generate the bar graphs indicating distances to an average cultured lung EC sample. Clustering was unsupervised except when only genes associated with the GO term “angiogenesis” were used. Pathway analysis was performed using the set of differentially expressed genes for an indicated comparison. Genes were considered differentially expressed if their log2 fold change was greater than or less than 1 for the comparison of their averaged FPKMs and if the p-value was <0.05 according to a two-sided t-test, and not assuming equal variance. Terms among the top 10 GOTERM_BP_FAT category, as determined by DAVID, were used (Huang da, W. et al., Nat Protoc 4, 44-57, (2009)). Heatmaps were generated using the pheatmap function in R after normalizing FPKM values by the maximum value for a given transcript.

### Genomic Stability

DNA from fresh/cultured cells was isolated and purified using PureLink Genomic DNA Mini Kit (Invitrogen K182001). DNA library preps were prepared and multiplexed and used as input for low coverage whole genome sequencing with HiSeq 100, producing 100 base pair single-end reads. Sequencing reads were de-multiplexed (bcl2fastq), checked for quality (FastQC), and trimmed/filtered when appropriate (Trimmomatic). The resultant high quality reads were mapped (BWA-MEM) to the UCSC mm9 genome build. Uniquely mapped reads were then binned (BEDTools) into 10K bp-sized stepwise, non-overlapping window tiles spanning the mm9 genome build sequence. The resultant read counts data was then used to generate segmentation profiles for chromosomes 1-19, X, and Y.

Counts were normalized to the total number of mapped reads per sample and scaled with the total number of reads in the reference sample. To ensure real value ratios, the scaled reads were adjusted by +1 count. Log2 ratios were acquired by performing log2-transformation of the adjusted and scaled experimental count over the adjusted and scaled reference count. To identify genomic regions with abnormal copy number, a circular binary segmentation (CBS) algorithm was implemented (Olshen, A. B. et al. Biostatistics 5, 557-572, (2004)) on the DNA copy number data to partition genomic regions that were divergent. The R-package DNAcopy to plot segmentation profiles (via CBS algorithm) was utilized for each sample in reference to LSECs. Default parameters were employed for all data processing commands

### Fibrin Bead Assay

Method was adapted for use with cultured mouse cells from Nakamatsu et al., (Nakatsu, M. N. et al., J Vis Exp, 186, (2007)). 5×10⁵ cells were incubated with 1260 Cytodex 3 Collagen beads (GE Life Sciences #17-0485-01) overnight at 37° in 5% CO2 in EC media. The next day, the cells/beads were washed and resuspended in 2 mg mL-1 fibrinogen (Sigma-Aldrich #F8630) in PBS with 0.15U mL-1aprotonin (Roche #10236624001). This mix was plated with 1.25U mL-1 thrombin (Sigma #82050-844) and allowed to form a solid matrix. Cell/bead/matrix preparations were cultured in StemPro-34 Serum Free Media (Invitrogen #10639-011), reconstituted according to the manufacturer's instructions and supplemented with 1× L-Glutamine (Thomas Scientific #B003L18), 1× Pen/Strep (Invitrogen #15240-062), 1× β-mercaptoethanol, 0.5 mM Ascorbic Acid (Millipore), 200 μg mL-1 bovine holotransferrin (Sigma Aldrich #T1283), 100 μg mL-1heparin (Sigma-Aldrich H3149), 20 ng mL-lmouse VEGF-A (Peprotech #450-32), and 20 ng mL-1FGF2 (Peprotech #100-18B). After 4 days, EC connections were scored by counting the number of matrix-suspended beads connected by cells and dividing by the total number of beads in a given field. Each experiment included 3 wells of cell/bead mixes and images were taken of each well, and the mean connected percentage across those wells were averaged to generate one n. Additional controls, which contained no thrombin, were used to confirm comparable cell survival across cell types and experiments.

### In Vivo Engraftment: Matrigel and Partial Hepatectomy

Cells were transduced with mCherry or GFP expressing lentivirus and purified by FACS. 2×10⁶ Cells were suspended in PBS containing 100 μg mL-1 heparin Sigma-Aldrich H3149), 20 ng mL-1 mouse VEGF-A (Peprotech #450-32), and 20 ng mL-1 FGF2 (Peprotech #100-18B), mixed 1:1 with Matrigel (BD Biosciences #354234), and injected subcutaneously into the flanks of C57BL/6J mice. 7 days later, mice were retro-orbitally injected with fluorophore-labeled anti-VEcad antibody and sacrificed. Plugs were removed, photographed, and, if being analyzed by microscopy, fixed in 4% paraformaldehyde then embedded in OCT. If incorporation was being quantitated, plugs were enzymatically digested with 2.5 mg mL-1 Collagenase A (Roche #11088793001) and 1 unit mL-1 Dispase II (Roche #04942078001) for 30 minutes at 37° C. under gentle agitation, filtered, and analyzed by flow cytometry.

For hepatectomies, the right medial, left medial, and left lateral lobes of C57B1/6 mice were resected with silk suture (Roboz) after anaesthetization with 100 mg kg-1 ketamine and 10 mgkg⁻¹ xylazine. The sutures were used to tie off the individual lobes, one at a time, and scissors were used to but the lobe just distal to the suture to minimize injury and blood loss. 5×10⁵ GFP or tdTomato-labeled cells were resuspended in PBS and injected intrasplenically into mice that underwent 70% partial hepatectomy, as described previously 20. After 14 days, mice were retro-orbitally injected with fluorescently-labeled anti-VEcad, sacrificed, and the organs were fixed, mounted, and prepared for imaging by fluorescent microscopy.

### Chromatin Immunoprecipitation and Analysis

Anti-Fli1 or control Rabbit IgG (Ab46540) were used to identify Fli1-bound regions using a method based on detailed protocol report 20. 2-5×10⁷ cells were fixed in 1% paraformaldehyde diluted in EC media. Fixation was quenched with 125 mM glycine and the cells were washed three times with PBS. After nuclei isolation and sonication using a Bioruptor, chromatin-protein complexes were incubated with 10 μg antibody bound to Dynabeads M-280 (Invitrogen) overnight at 4° C. under gentle agitation. Complexes were washed with PBS containing 0.5% BSA and 5 mM EDTA using magnetic separation and then DNA purified by phenol-chloroform extraction. Enrichment was tested by QPCR, paired-end (75/75 bp) libraries were produced and then sequenced at the MSKCC Integrated Genomics Operation on Illumina HiSeq 4000. Sequences were mapped to the mm9 genome reference using bwa mapper. Analysis of Fli1 DBRs was done using a combination of SAMtools and custom R and Python scripts to carry out general linear modeling (Robinson, M. D. et al., Bioinformatics 26, 139-140, (2010)). Salient features of this analytical framework are the use of a negative binomial error model and appropriate false discovery rate corrections to identify Fli1 DBRs associated with a particular phenotype. This method controls the type 1 error while preserving good detection power for differential binding. Contrast models were used to identify differential Fli1 sites in the different cell types. DBRs were prioritized by the fold change between groups (Fli1 v IgG or between cell types) and the corrected p-value, to identify the genomic elements with the most robust group effect on the Fli1 binding purview. Motif analyses were performed with tools from the MEME suite (Bailey, T. L. et al., Nucleic Acids Research, 37, W202-208, (2009)).

### Hindlimb Ischemia

The proximal part of the femoral artery and the bifurcation point between the popliteal artery and the saphenous artery of C57B1/6 mice were ligated and all side braches were dissected. The femoral artery was excised out via ligated points. With the muscle still exposed, 5×10⁵ cells suspended in PBS were injected into the gracilis muscle. Hindlimb reperfusion was measured at indicated time points with laser Doppler perfusion imager (Persican PM3, Perimed). For quantification or visualization, one randomly selected member of each group was selected and, retrorbitally injected with fluorescently-labeled anti-VEcad antibody and sacrificed to visualize tissue. Fixed and mounted tissue sections were analyzed by fluorescent microscopy.

### Statistics

Significance of pairwise comparisons were determined using unpaired Student's ttests with a significance threshold at P<0.05. All values are presented as mean with error bars indicating standard deviations. For genomic stability, a cutoff threshold of 3+ absolute standard deviations was used for segment split calling. For ChIP-seq, selection criteria were set at absolute value (log fold change) >1.5, FDR <0.01, logCPM >-3.

### Data Availability

Sequencing data have been deposited in the The National Center for Biotechnology Information GEO repository accession code GSE85642.

### Example 2

### Conversion of Mouse Amniotic cells to EC-Like Cells

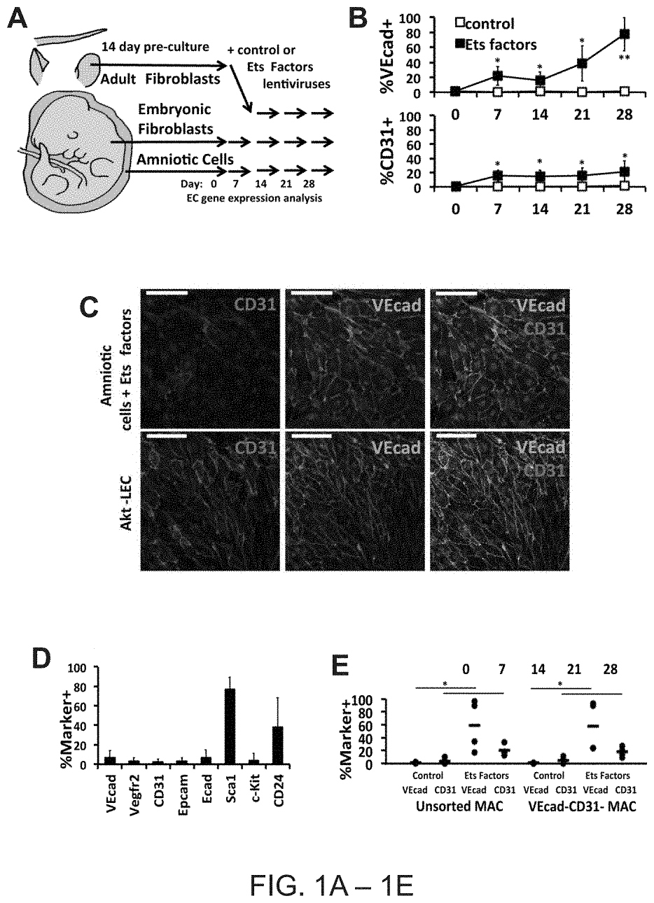

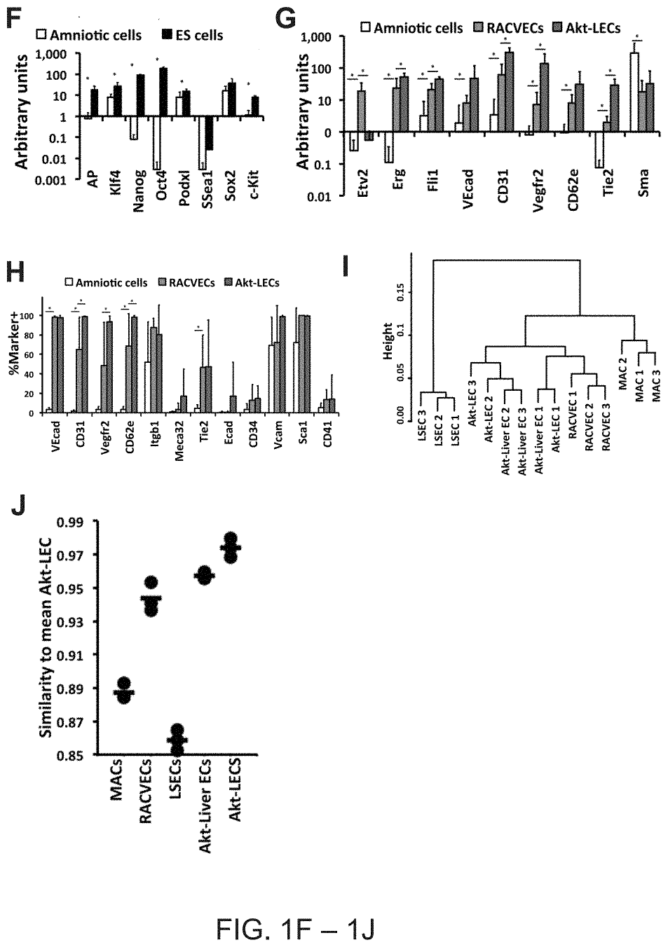

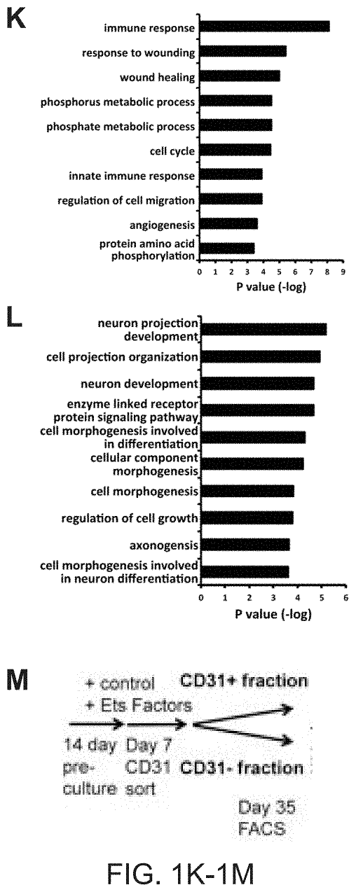

In order to characterize the vascular and regenerative function of reprogrammed EC-like cells, well-defined congenic mouse models were employed that overcome the confounding influence of xenografting immune-compromised mice (e.g., NSG) and the use of genetically disparate human cell sources. Mouse amniotic cells (MACs) were harvested from E11.5-E13.5 C57BL6/J embryos and were transduced with lentiviruses encoding mouse Ets TFs, Etv2, Erg, and Fli1 and the transduced cells were propagated using EC culture conditions and a TGF-β signaling inhibitor. Empty null lentivirus constructs were used as negative controls. In parallel, mouse embryonic fibroblasts (MEFs) collected from E13.5 embryos and mouse adult fibroblasts (MAFs) collected from adult tail and ear tissue (FIG. 1A) were also converted. Expression of the Ets TFs after transduction was similar in all three cell types as assessed by western blot and qPCR. All cell types transduced with Ets TFs lentiviruses expressed EC-linked transcripts at some point during conversion. As the converting cells upregulated EC-associated transcripts during conversion, they also reduced non-EC genes such as the fibroblast gene Cspg4 (MAFs) and smooth muscle acting, Sma (MEFs, MACs) (. Reduced Sma expression in Ets TF transduced MEFs was observed only at day 21. Hence, all three-cell types tested could acquire some EC features.

Surface protein expression of VEcad and CD31 was confirmed by flow cytometry in all three cell types transduced with Ets TF-expressing lentiviruses but not in the corresponding controls (FIG. 1B). The proportion of converted MACs expressing VEcad (≥80% ) and CD31 (≥20% ) gradually increased by day 28 (FIG. 1B). Adult mouse lung ECs (Akt-LECs) were also cultured by transducing them with a constitutively active Akt-signaling to facilitate propagation ex vivo, in part, by preventing apoptosis through simulation of the shear forces ECs experience in vivo(Poulos, M. G. et al., Stem Cell Reports, (2015); Israely, E. et al., Stem Cells, 32, 177-190, (2014); Dimmeler, S. et al., Circulation Res., 83, 334-341 (1998); Dimmeler, S. et al. Nature, 399, 601-605, (1999); Fujio, Y. & Walsh, K., JBC, 274, 16349-16354 (1999); Nolan, D. J. et al., Dev Cell, 26, 204-219, (2013); Sodhi, A. et al., PNAS, 101, 4821-4826, (2004)) Immunofluorescence (IF) staining of Akt-LECs and Ets-transduced cells showed VEcad protein loaded onto the cell surface where it coalesced at cell-cell junctions, as is characteristic of primary adult ECs (FIG. 1C). MAC-derived cells adopted the shape and morphology of Akt-LECs and co-expressed surface CD31 in a subset of converted cells (FIG. 1C). Control MAC cultures did not spontaneously convert to EC-like cells (FIG. 1B). For MAF- and MEF-derived cells, expression of both VEcad and CD31 plateaued between days 7 and day 28. By microscopy these cells expressed low levels of VEcad and CD31 on their surface. Thus, the converted MAF and MEF cells were either being outcompeted by non-converted cells or they could not maintain an EC-like phenotype during in vitro expansion.

To test if the failure of EC-like MAF- and MEF-derived cells to stably expand was due to outgrowth by unconverted cells, transduced cells were sorted on day 7 based upon their expression of CD31. The sorted cells were then propagated for an additional four weeks and re-analyzed expression of the EC markers to examine if the progeny retained the sorted immunophenotype (FIG. 1M). The CD31-positive and negative MAF fractions became indistinguishable over time. Only half of the MEFs remained CD31⁺ by day 28 and a small number of CD31-neg MEFs acquired CD31 expression and over time. In contrast, MAC-derived CD31⁺ cells maintained their EC character with persistent expression of CD31 and VEcad and the CD31⁻ MAC progeny never acquired these EC-like features (FIG. 1M). Therefore, conversion of adult and fetal fibroblasts was erratic and unstable whereas MACs attained and stably maintained EC-like characteristics after transduction with the Ets TFs.

Next step was to ensure that the stable conversion of MACs to an EC-like cell fate was not due to the selective outgrowth of a contaminating EC or pluripotent populations. It was found that MAC preparations contained small amounts of VEcad, CD31, and Vegfr2 transcripts likely due to a small number of cells expressing endothelial markers (FIG. 1D). To exclude the possibility that MAC-derived VEcad⁺ or CD31⁺ cells were derived from pre-existing cells marked by EC proteins, MACs were depleted of cells expressing CD31 or VEcad and found that the non-vascular MACs could be converted just as well as the unsorted population (FIG. 1E). Significant c-kit⁺ cells could not be detected, but it was found that some MACs expressed Scal and CD24, indicating they might express other pluripotent genes (Shakiba, N. et al., Nature communications, 6, 7329, (2015).). But comparison of transcript levels of pluripotent genes in MACs to embryonic stem cells showed that MACs were largely devoid of pluripotency-associated transcripts (FIG. 1F). Therefore, these data indicate that we have directly converted non-pluripotent and non-vascular cells into EC-like cells.

Next, the molecular and functional characteristics of the converted cells were comprehensively defined. Because instability of converted embryonic and adult fibroblasts precluded such analysis, the converted MACs were mainly focused on. Since mouse MACs transduced with Etv2, Erg, and Fli1 and propagated in EC growth conditions for 28 days were similar to human RACVECs in their unique EC stability, henceforth they are referred to as RACVECs. The transcriptomes of MACs, RACVECs, and cultured adult mouse LECs expressing constitutively-active myristoylated Akt were compared. MACs and cultured Akt-LECs expressed much less Etv2 than RACVECs (FIG. 1G). All of the adult EC genes tested were strongly induced in RACVECs compared to MACs (FIG. 1G). In RACVECs, the expression levels of some of these genes (VEcad, CD31, and Vegfr2) were indistinguishable from Akt-LECs, whereas others (Erg, Fli1, CD31, CD62e, and Tie2) were not as highly expressed, or more variable across isolates, compared to Akt-LECs. Non-vascular specific genes, such as Sma, were downregulated in RACVECs to levels comparable to Akt-LECs. Transcript levels were corroborated by analysis of EC protein expression by flow cytometry (FIG. 1H). Thus, murine RACVECs have stably adopted many, but not all, morphologic, transcriptional and immunophenotypic features of ECs.

The mRNA of MACs, RACVECs were sequenced. Cultured ECs and as an additional control, non-lymphatic ECs directly purified from mouse livers , referred to as LSEC, which were a readily available abundant source of non-cultured ECs, were sequenced as well. It was found that RACVECs clustered more closely to cultured ECs than they did to MACs (FIG. 1I). The distances from the clustering analysis were extracted and it was observed that RACVECs were more similar to cultured primary LECs than to MACs (FIG. 1J). Differences in gene expression between cultured and directly purified cells were paramount. This separation was not apparent when the clustering was restricted to genes associated with the GO term “angiogenesis,” suggesting that our cultured cells maintained the essential characteristics of ECs and the differences between cultured and directly purified cells were attributable to their disparate environments. Since the goal was to analyze cells that can be propagated in vitro for eventual transplantation, the analysis was focused on differences among the cultured cell types. On average, 624 genes were upregulated in RACVECs compared to MACs and pathway analysis indicated that this group was enriched for EC-associated functions and angiogenesis (FIG. 1K). Moreover, 674 genes were downregulated in RACVECs compared to MACs, and for this group, the most enriched pathways were neuron projection development and morphogenesis involved in differentiation (FIG. 1L). Genomic sequencing was performed of cultured MACs, RACVECs, and directly purified LSECs and, similar to human amniotic cells and human RACVECs, no gross genetic alterations in the cultured cells were identified compared to the directly purified adult ECs, indicating genomic stability. Hence, RACVECs broadly and thoroughly adopted an EC-like identity and conversion erased the original MAC identity.

### Example 3

### Akt-signaling Enhances RACVEC Endothelial Function

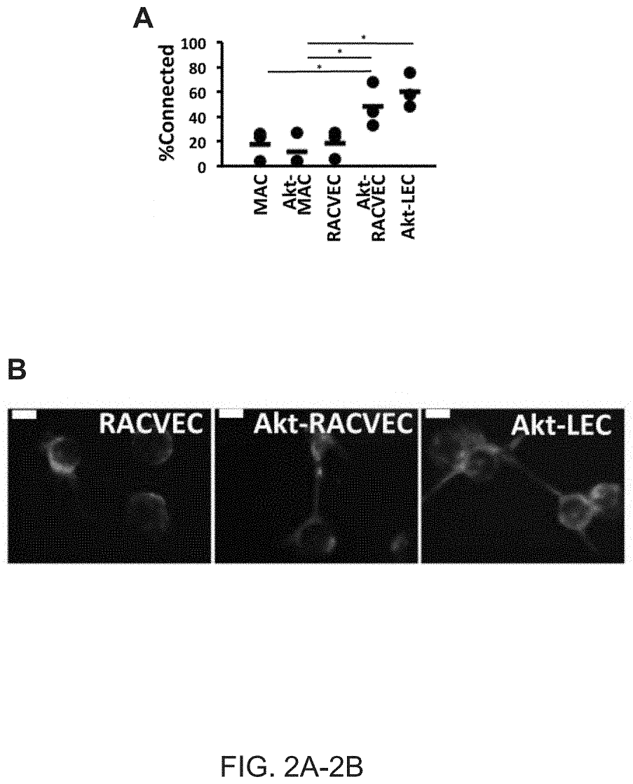

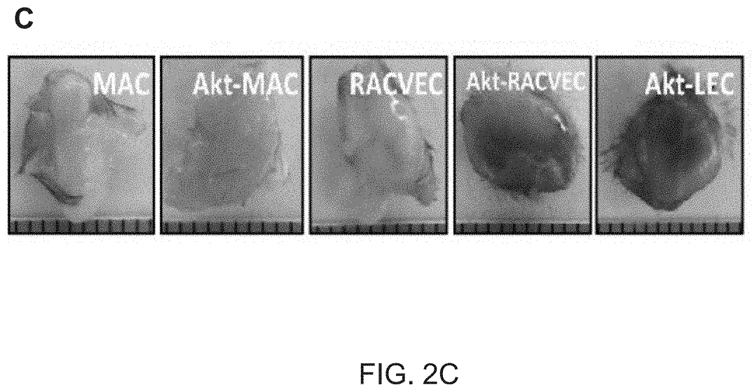

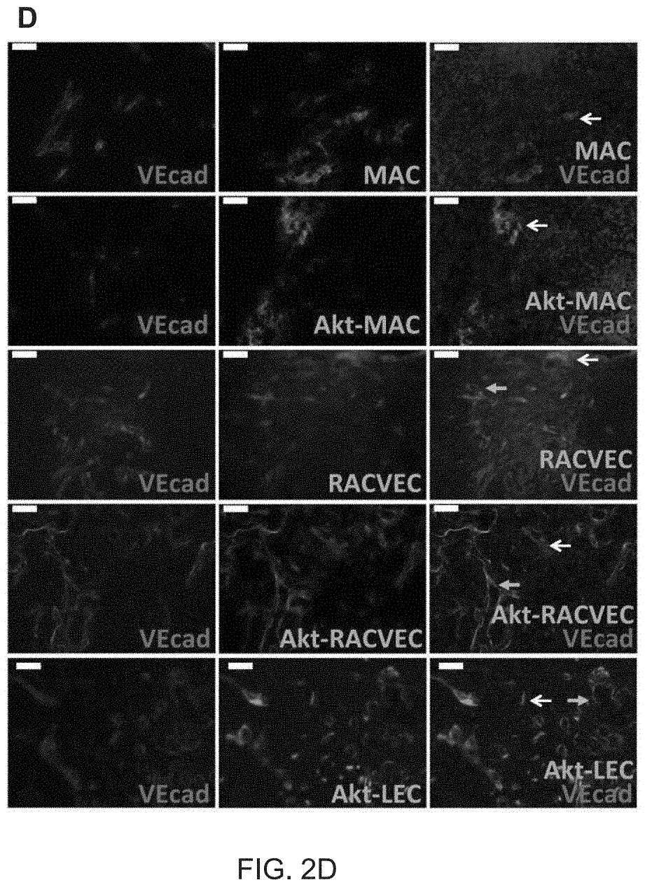

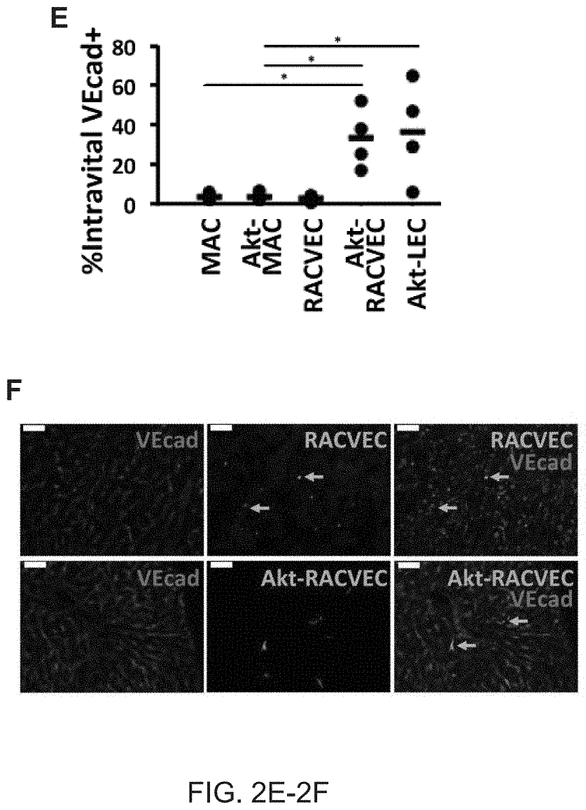

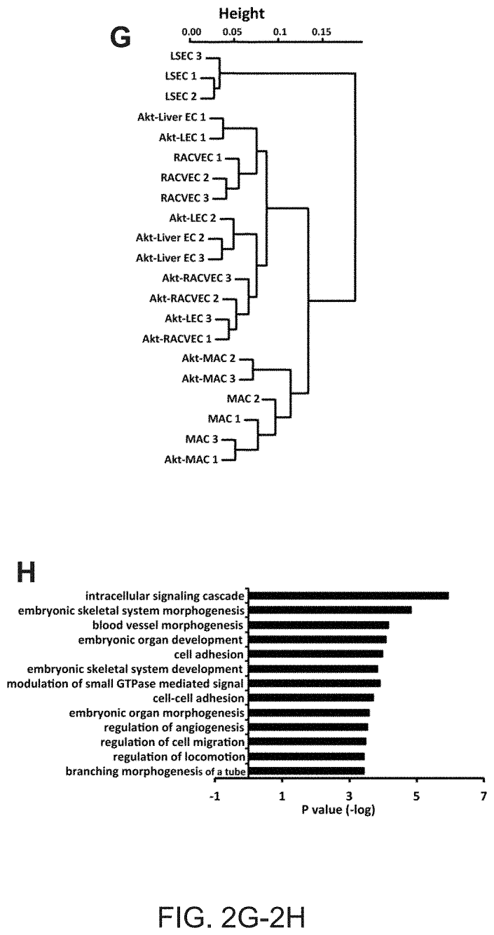

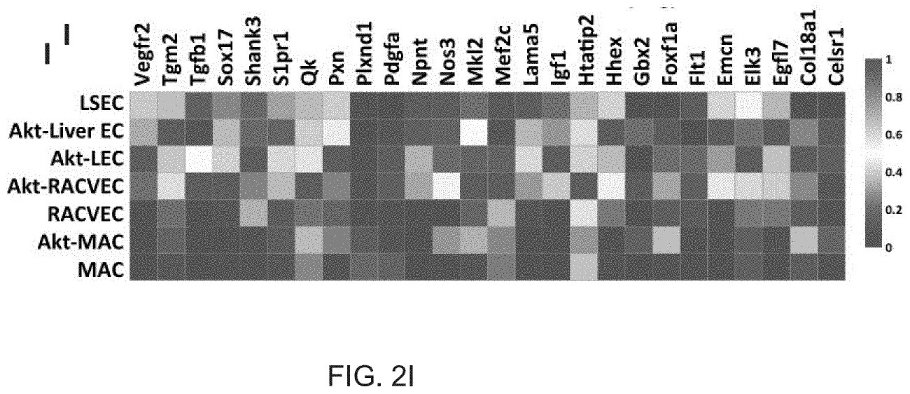

The ultimate goal is to generate abundant, stable vascular cells that can form new vessels after transplantation and engraft into existing vessels. However, clear interpretation of human RACVEC functions in vivo was unavoidably obstructed by donor cell heterogeneity and the technical requirement for xenograft analysis. Accordingly, the ability of RACVECs to form vascular networks in vitro was tested using a fibrin bead assay. RACVECs formed as few connections as MACs and less than Akt-LECs (FIGS. 2A-2B). Thus, it appeared that despite the phenotypic and molecular similarity of RACVECs to primary ECs, the converted cells were missing key programs necessary for vascular functions.

Constitutively active Akt-signaling enables stable propagation of functional ECs, thus, it was tested whether this approach could confer vascular functions to RACVECs. RACVECs transduced with a lentivirus driving expression of myristoylated Akt (Akt-RACVECs) could be stably propagated in vitro, as assessed by morphological, and immunophenotypic analyses. Akt-signaling did not alter the transcript levels of the Ets factors. Akt-RACVECs and RACVECS did not express high levels of genes associated with tumor EC phenotypes, compared to control cultured ECs (Seaman, S. et al., Cancer Cell, 11, 539-554, (2007)) .

To determine if Akt-signaling improved the EC function of RACVECs, in vitro surrogates and direct in vivo tests were used. Unlike RACVECs, Akt-RACVECs readily formed connections in vitro (FIG. 2A-2B). Matrigel plugs loaded with GFP-marked MACs, Akt-MACs, RACVECs, Akt-RACVECs, or Akt-LECs were injected subcutaneously into the flanks of C57B1/6 mice. After 7 days, fluorescently labeled anti-VEcad antibody was retro-orbitally injected to visualize functional, perfused vessels (intravital VEcad⁺). Upon removal of the plugs, it was found that only the matrigel grafts with Akt-RACVECs and Akt-LECs were perfused with blood, as indicated by reddish coloring of recovered material (FIG. 2C). Examination of the plugs by fluorescent microscopy showed that Akt-RACVECs and Akt-LECs readily engrafted into vessels anastomosed with the recipient vasculature (GFP⁺ intravital VEcad⁺), whereas the vast majority of RACVECs were unincorporated (GFP⁺ intravital VEcad⁻) into host vessels (FIG. 2D). The plugs were digested to quantify the percentage of injected cells integrated into the recipient circulatory system. RACVECs displayed minimal incorporation (appearing as GFP⁺ intravital VEcad⁻), similar to negative controls MAC and Akt-MAC. In contrast, Akt-RACVECs and Akt-LECs incorporated into and anastomosed with the host vasculature (GFP⁺ intravital VEcad⁺) (FIG. 2E). A model of organ regeneration was used, in which 70% partial hepatectomy triggers compensatory liver regrowth requiring neo-angiogenesis and angiocrine support (Ding, B. S. et al., Nature, 468, 310-315, (2010); Hu, J. et al., Science, 343, 416-419, (2014)). While both Akt-RACVECs and RACVECs could be identified in the regenerating liver, only Akt-RACVECs were incorporated into vasculature of regenerating livers as reported by intravital VEcad staining (FIG. 2F). Hence, Akt-RACVECs, but not RACVECs, performed morphogenic functions necessary for EC tube formation and engraftment into recipient blood vessels.

### Example 4

### Akt-Signaling Modulates RACVEC Gene Expression

To assess how constitutively active Akt-signaling functionalizes RACVECs, the transcriptomes of MACs, RACVECs, Akt-MACs, Akt-RACVECs, Akt-LECs, murine iver ECs (Akt-Liver ECs), and directly purified liver ECs were compared. Constitutively-active Akt-signaling did not make the global transcriptional profile of MACs or RACVECs more similar to adult primary ECs, as assessed by K-means distance and principle component analysis (FIG. 2G). The relative similarity of Akt-RACVECs to Akt-LECs or Akt-Liver ECs was no greater when the analysis was restricted to genes within the “angiogenesis” gene ontology and directly isolated ECs clustered with cultured EC and converted cells. Analysis of endothelial TFs and EC markers showed that some genes were differentially expressed, but there was no obvious pattern. Genes differentially expressed in the cultured cells were focused on, because the goal was to identify factors contributing to in vitro stable propagation and eventual transplantation. Within the set of genes more highly expressed in Akt-RACVECs than RACVECs were genes associated with adhesion, and vessel and tube morphogenesis, consistent with the functional deficiencies observed in RACVECs (FIG. 2H). Pathways enriched in the set of Akt-downregulated genes revealed that adhesion genes were also enriched, reinforcing the notion that Akt-signaling alters the ability of converted cells to stably connect with ECM and other cells. The relative expression level of differentially regulated genes within the morphogenesis-associated ontologies is shown in FIG. 21. Among these genes upregulated were EC TFs, specifically Mef2c, Tal1, Elk3, Hhex, and Sox17, as well as matrix and receptor-signaling proteins Col1al, Emcn (Endomucin), Shank3, and Vegfr2, and CD31. Thus, Akt-RACVECs are broadly similar to RACVECs, but activate EC genes that confer EC tubulogenic and morphogenic functions.

### Example 5

### Akt-Signaling Modifies the Fli1 Genomic Binding in RACVECs

In contrast to ES-derived and adult ECs, Akt-signaling was not required for stable RACVEC culture, and as such, RACVECs could be used to parse the endothelialfunctions of Akt-signaling separate from its pro-survival effects in vitro.

ChIP-seq was used to directly test the hypothesis that Akt-signaling might support in vivo function by modifying the genomic Fli1 binding site purview to enrich EC gene targeting and extinguish binding to nonvascular genes. The Fli1 genomic binding purviews in RACVECs, Akt-RACVECs, Akt-LECs, and freshly isolated liver ECs were compared by ChIP-seq. With this approach ˜11,000 of Fli1 differentially-bound regions (DBRs) that were shared by all cell types were identified, likely representing a “Core” endothelial signature (FIG. 3A). These core DBRs typically occurred near 5′ regulatory regions of annotated refGene transcripts (FIG. 3B, left). Data from the ENCODE project (Rosenbloom, K. R. et al., Nucleic acids research, 41, D56-63, (2013)) was utilized to map EC regulatory regions identified in HUVEC to the mouse genome and found that Fli1 DBRs occurred near EC promoter and enhancer regions (FIG. 3B, right) including genomic sites near VEcad, CD31, Vegfr2, genes expressed in ECs and activated during the conversion process (FIG. 3C). Thus, the Fli1 binding purviews in converted cells is highly enriched for regions involved in vascular gene regulation.

While clustering and PCA analyses of transcriptional profiles indicated that Akt-signaling did not broadly enhance EC gene expression, it was found that Akt-signaling refined the Fli1 genomic binding site purview of RACVECs making it more similar to Akt-LECs and LSECs (FIG. 3D). Whereas Akt-RACVECs shared ˜90% of their Fli1 binding sites with Akt-LECs and ˜64% with LSECs, only about 67% of the DBRs in RACVECs were shared with Akt-LECs and ˜35% with LSECs. The increased similarity with freshly isolated LSECs indicated that the genomic purview change observed was not simply a result of myr-Akt, but reflective of the ability of Akt-signaling to simulate the in vivo environment.

To uncover how constitutive Akt-signaling increased the similarity of the Fli1 purview in RACVECs, datasets composed of sites differentially bound by Fli1 in Akt-RACVECs ompared to RACVECs were generated. These sets of DBRs were called “Akt-RACVEC Up,” and “Akt-RACVEC Down” (FIG. 3E). The Akt-RACVEC Down set was further restricted, and a “RACVEC Unique” set composed of ˜7,000 sites that were downmodulated by Akt-signaling in RACVEC (i.e., Akt-RACVEC down) and also not enriched in either Akt-LEC or LSEC was created (FIG. 3E). DBRs belonging to the Akt-RACVEC Up set were bound by Akt-LECs and LSECs, supporting the notion that Akt-signaling mimics the native EC environment (FIG. 3E). Three derived chromatin state maps were generated: genomic regions marked with a defining chromatin state in any cell type (Composite); regions marked by a chromatin state in HUVEC, but not other cell types (HUVEC Unique); and regions marked by a chromatin state in cell types other than HUVEC (HUVEC Excluded). It was discovered that the Core EC, RACVEC unique, and Akt-RACVEC Down sites were associated with promoter and enhancer regions found in all of the cell types analyzed (FIG. 3F) (Ernst, J. et al., Nature, 473, 43-49, (2011)). RACVEC Unique and Akt-RACVEC Down sites were enriched at genomic regions that were marked as promoter and enhancer elements in non-HUVEC (FIG. 3F). In contrast, sites belonging to Akt-RACVEC Up were associated with regions uniquely marked as promoters or enhancers in HUVEC (FIG. 3F, HUVEC Unique) and relatively excluded from regulatory regions absent in HUVEC (FIG. 3F, HUVEC Excluded and other cell types). The overlap of sites in these sets with the human Fli1 purview in HUVECs in Patel et al. (Patel, M. et al., Genome Res, 22, 259-270, (2012)) was also analyzed, and it was found that the Core and Akt-RACVEC Up sets overlapped with murine regions that corresponded to regions identified as human Fli 1 targets far more likely than by chance (FIG. 3G). Therefore, Akt-signaling extinguishes Fli1 binding to extraneous binding sites and drives binding to genomic sites key to EC identity and function.

To identify the TFs that might bind to key EC regulatory regions, de novo motif discovery was performed using the genomic sequences of Fli1 DBRs identified in each cell type and dataset and searched for known motifs in the JASPAR database (Bailey, T. L. et al., Nucleic acids research, 37, W202-208, (2009)). As expected, a canonical GGAA Ets binding motif was found within all Fli1 DBRs and the most highly preferred Ets-like motif, (A/C)GGAA(G/A), was very similar in all cell types and DBR sets studied. CCAAT box motifs were preferentially enriched with Fli1 DBRs in RACVECs and in the RACVEC unique set, but not in the other cell types or in the set of Core Fli1 DBRs (FIG. 3H). In contrast, a Sox consensus motif was strongly enriched in Fli1 DBRs found in Akt-LECs and Akt-RACVECs but not in DBRs found in RACVEC Unique. Furthermore, an unbiased search for accessory motifs near the most highly enriched Ets motif identified Ebox and Sox sites in the Akt-RACVEC Up set. Finally, Sox sites were enriched in the sequences in the regions differentially bound by Fli1 in the RACVECs versus Akt-RACVECs. Thus, Akt-signaling modifies Fli1 genomic binding site selection, favoring regions unique to ECs that often contained Sox motifs.

Because multiple Sox family members have been implicated in EC development and function (De Val, S. & Black, B. L., Dev. Cell, 16, 180-195, (2009)), the inventors searched for a specific Sox factor that might be associated with the Sox binding motif. The relevant Sox factor should be expressed in Akt-RACVECs and Akt-LECs but not in RACVECs. The inventors found that only Sox17 met these criteria, suggesting that Akt-signaling both induced Sox17 expression and repositioned Fli1 genomic binding towards Ets sites with nearby Sox motifs (FIG. 3I). Western blotting confirmed that Sox17 protein was present in Akt-RACVECs, Akt-LECs, and LSECs, but absent in RACVECs. These results raised the possibility that Sox17 could replace Akt-signaling to enhance EC morphogenic function in RACVECs and promote engraftment.

### Example 6

### Sox17 Augments RACVEC EC Function

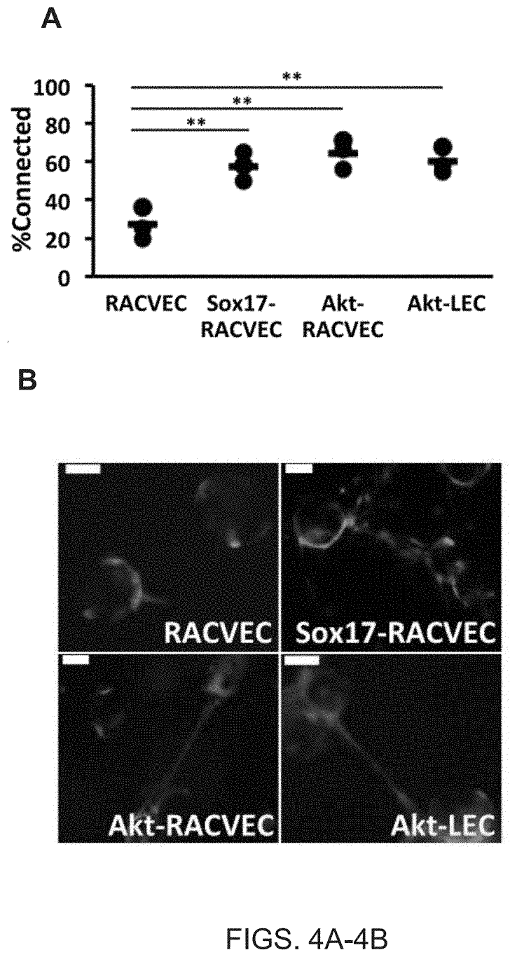

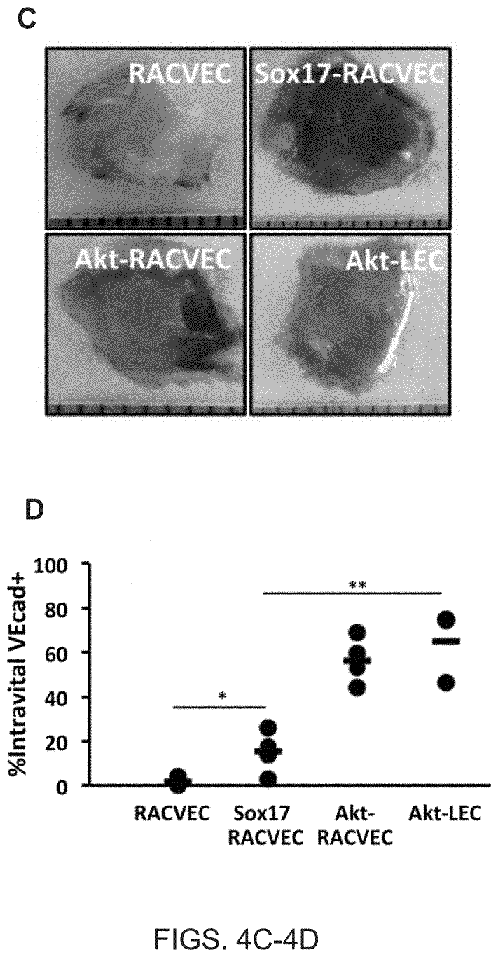

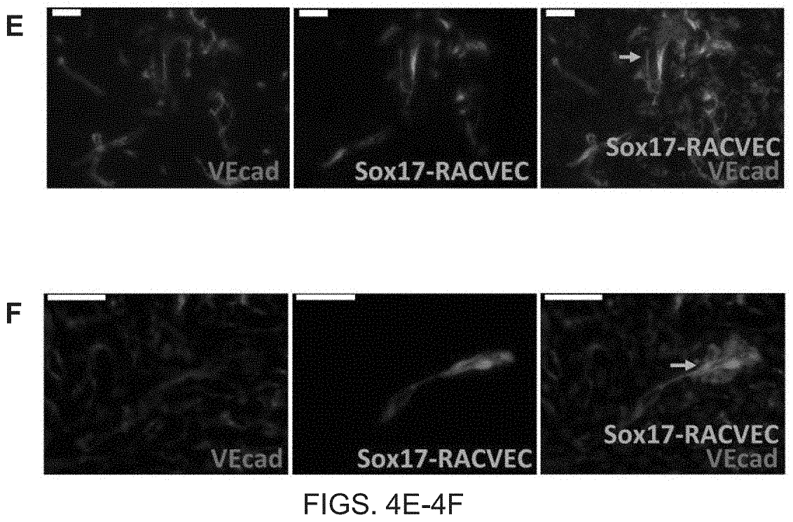

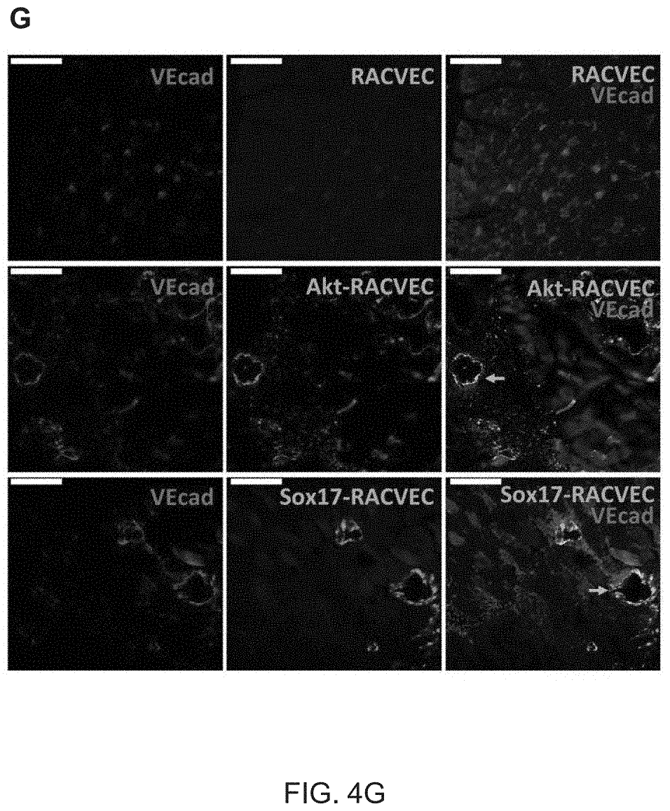

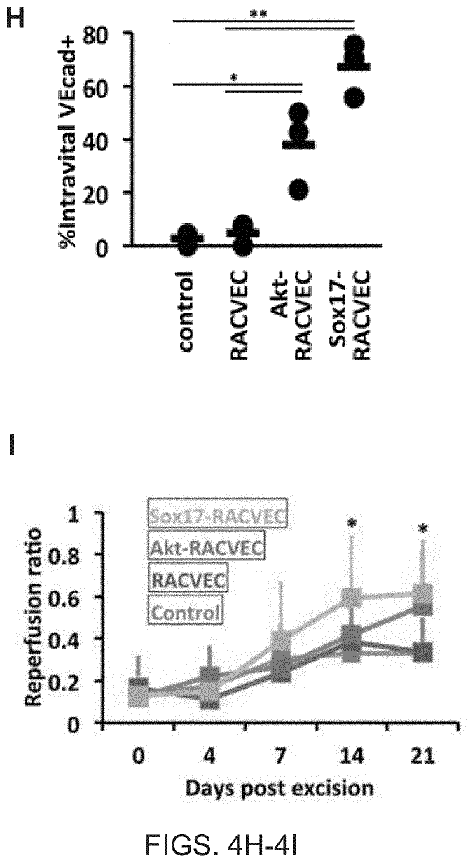

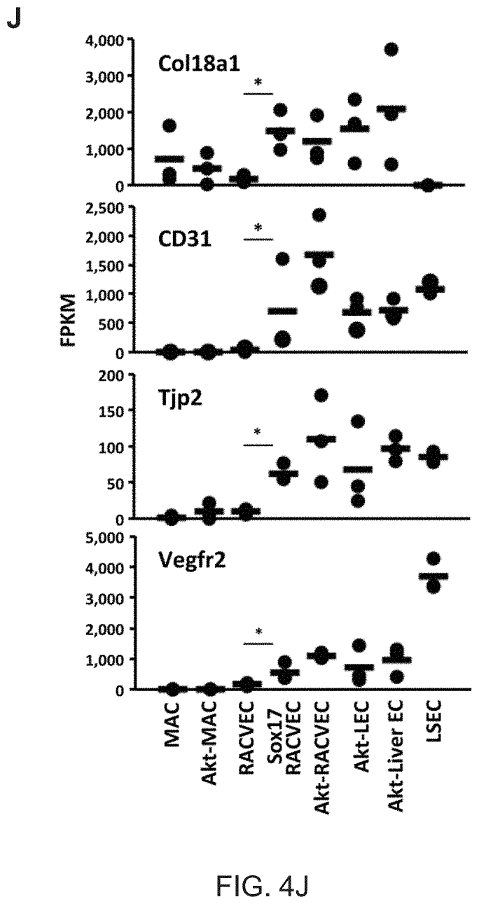

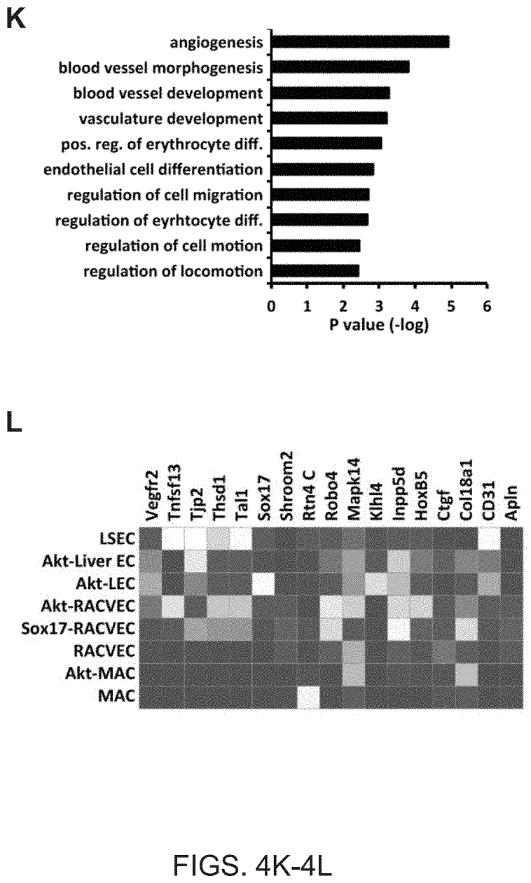

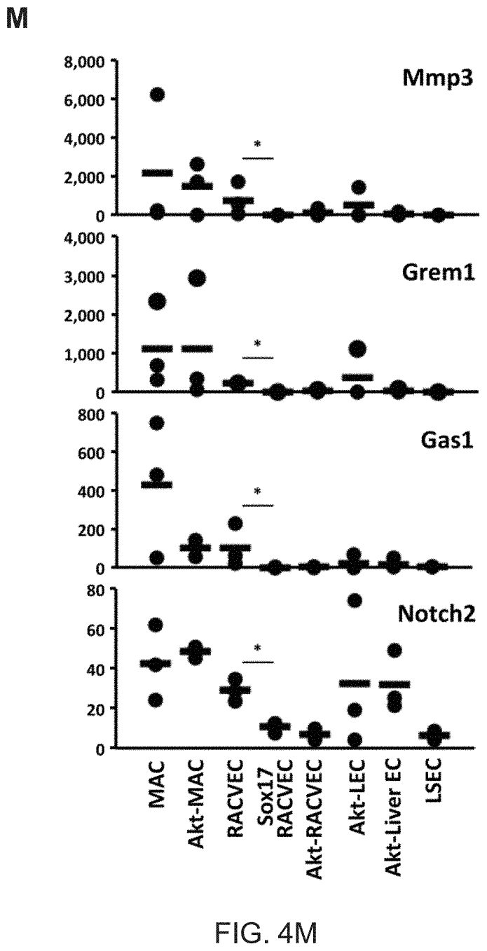

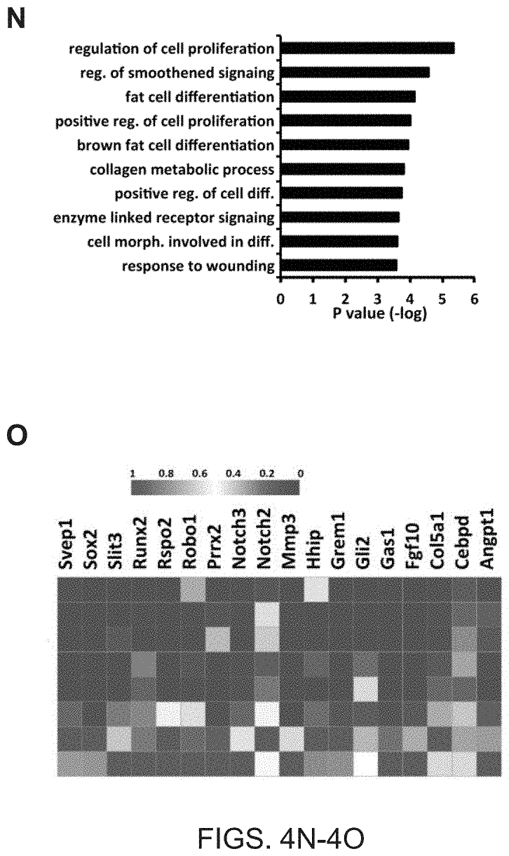

The inventors hypothesized that enabling Sox17 expression in RACVECs could replace constitutively active Akt-signaling and rescue the defects of RACVECs. To test this, expression of Sox17 was enforced in RACVECs and whether this could rescue RACVEC defects using the fibrin and matrigel plug vascularization assays was tested. It was found that enforced Sox17 expression in RACVECs (Sox17-RACVECs) enhanced in vitro EC vascular function (FIG. 4A-4B). Similarly, Sox17-RACVECs engrafted into lumenized, anastomosed vessels perfused with blood in matrigel plugs or defined matrix such as fibrin (FIG. 4C-4E). Similarly, Sox17-RACVECs engrafted into lumenized intravital VEcad⁺ blood vessels when transplanted into mice after partial hepatectomy (FIG. 4F).

To assess engraftment and function of Sox17-RACVEC in an assay of revascularization, GFP-marked RACVECs, Akt-RACVECs, Sox17-RACVECs or saline (control) were injected intramuscularly after partial femoral artery excision. Cells exposed to circulation were intravitally-labeled by injecting labeled anti-VEcad antibody and found that at day14 Akt-RACVECs and Sox17-RACVECs were incorporated into host vasculature (FIG. 4G). The microscopy results were corroborated using flow cytometry of digested day 14 thigh muscle tissue (FIG. 4H). While transplanted Akt-RACVECs and Sox17-RACVECs displayed engraftment, only Sox17-RACVECs enhanced reperfusion by days 14 and 21 compared to control (FIG. 4I). Unincorporated RACVECs were observed at day 1 after surgery indicating that their absence at later times might be due to their inability to stably incorporate into vessels. Indeed, failure to recover RACVECs at later stages could be due to passive dispersal of cells or cell death as a consequence of their failure to establish homotypic and/or ECM interactions (Corada, M. et al., Nature Comm., 4, 2609, (2013)). By contrast, Sox17-RACVEC and Akt-RACVEC engraftment was long-lasting and vessel-integrated cells could be observed two months after transplantation. Hence, Sox17 functionalizes transcriptionally converted EC-like cells so they can perform as stable bona fide blood vessel ECs after transplantation.

The transcriptomes of Sox17-overexpressing RACVECs were compared to our other EC populations. 141 genes were more highly expressed in Sox17-RACVECs than RACVECs and 30% of these genes were also found in the constitutive Akt-induced set. The set included Col18al, CD31, Tjp2 (ZO-2), and Vegfr2 (FIG. 4J). Similar to the genes induced by Akt-signaling, the Sox17-induced group was enriched for morphogenesis-associated genes (FIG. 4K). The relative expression of selected morphogenesis-and angiogenesis-associated gene are shown in FIG. 4L. It was found that 337 genes were downregulated, and 30% of these genes were also found in the set of genes that were reduced in Akt-RACVECs. The most strongly reduced genes included Mmp3, Grem1, Gas1, and Notch2 (FIG. 4M). While the genes downregulated by Akt-signaling were associated with cellular interactions and metabolic processes, those downregulated by Sox17 were associated with proliferation and Hedgehog signaling (FIG. 4N). Genes associated with most significantly enriched pathways are shown in FIG. 40. Thus, Sox17 modifies a group of genes that overlaps with those affected by constitutive Akt-signaling, and is enriched with genes associated with morphogenesis, cellular interactions, and proliferation.

Engineered ECs that can be expanded, transplanted, and engrafted into compromised blood vessels to restore their perfusion and instructive functions have the potential to significantly alter how vascular diseases are studied, treated and cured (Ramasamy, S. K. et al., Trends Cell Biol, 25, 148-157, (2015); Gulati, R. & Simari, R. D.; Dis Model Mech, 2, 130-137, (2009)). Since ECs play essential roles in organ development and regeneration, engineered engraftable ECs could help repair or replace injured tissue (Ding, B. S. et al., Cell, 147, 539-553, (2011); Takebe, T. et al., Nature, 499, 481-484, (2013); Hu, J. et al., Science, 343, 416-419, (2014); Poulos, M. G. et al., Stem Cell Rep., (2015)) or even reverse disease (Follenzi, A. et al., JCI, 118, 935-945, (2008); Hayashi, T. et al., Cornea, 27, 699-705, (2008); Brodsky, S. V. et al., Am J Physiol Renal Physiol, 282, F1140-1149, (2002)). While specific human ECs, such as HUVECs, can be propagated in culture, murine ECs tend to drift to nonvascular cells and cease proliferating. For this reason, the approach disclosed in this application of direct conversion of mouse mid-gestation amniotic cells to ECs compares favorably to previous reports of EC production from fibroblasts and pluripotent sources because of the stability and efficiency of RACVEC generation. Direct conversion of mouse adult fibroblasts to ECs indicated that adult fibroblast-derived ECs are unstable and do not maintain their EC identity over time (Han, J. K. et al., Circulation, 130, 1168-1178, (2014)). Human fibroblasts (Morita, R. et al., PNAS, 112, 160-165, (2015)) and adipose tissue (Fontijn, R. D. et al., Stem Cell Res, 13, 367-378, (2014)) can be converted to EC-like cells by Etv2 and Sox18, respectively, but xenobiotic barriers limited analysis of the converted cells to in vitro assays and short-term tests in immunocompromised mice. Thus, vascular durability in vivo could not be assessed in these studies. Many groups have explored the potential of generating ECs from human and mouse pluripotent cells or employing pluripotency factors (Margariti, A. et al., PNAS,109, 13793-13798, (2012); Li, J. et al., Arteriosclerosis, thrombosis, and vascular biology, 33, 1366-1375, (2013); Prasain, N. et al. Nature Biotechnology, 32, 1151-1157, (2014); Israely, E. et al. Stem Cells, 32, 177-190, (2014); Kurian, L. et al., Nat Methods, 10, 77-83, (2013)). RACVEC lineage stability may be a consequence of a gene or set of genes expressed, or not expressed, in amniotic cells, compared to fibroblasts. Alternatively, RACVEC stability may be attributable to a favorable chromatin state that allows TF access to a restricted set of stabilizing genes but not to genes that lead to fate instability/drift.

Transcriptome analysis of basic Ets-converted RACVECs indicated that they activated a broad range of genes expressed in cultured ECs and were more similar to cultured ECs than to their amniotic starting cell identity. RACVECs performed EC functions poorly, suggesting that endothelial functions require expression of genes not induced after enforced overexpression of Ets TFs and/or dynamic cellular adaptations that were not supported in the converted cells. In this disclosure, the inventors took advantage of the finding that microenvironment cues activate Akt to identify Sox17 as a factor that promotes engraftment, despite its being dispensable for broad EC gene activation.

The genomic targeting of Fli1 in converted cells was broadly similar to Fli1-binding in cultured and freshly purified ECs. Deeper analysis uncovered a novel role for constitutive Akt-signaling to refine the Fli1 genomic purview, shedding non-EC genomic targets in favor of a more minimal EC purview. Fli1 sites shed in the presence of Akt-signaling were enriched for CCAAT-box binding proteins (CEBP family members) that are more closely associated with hematopoietic and adipocyte activities. Since Fli1 is essential for hematopoietic function, enforced expression of Fli1 may drive it to bind promiscuously to many accessible Ets motifs, regardless of whether they regulate EC or non-EC genes. It is plausible that Akt-signaling shifts Fli1 binding either by altering affinity of Fli1 for particular Ets motifs or by shifting the repertoire of accessory factors with which Fli1 can bind.

Akt-signaling activated Sox17 expression and enriched Fli1 binding sites with nearby Sox motifs. Sox17 is more highly expressed by arterial cells that experience high shear (Corada, M. et al., Nature Comm., 4, 2609, (2013)), and by and tip cells (Lee, S. H. et al., Circulation Res. 115, 215-226, (2014)). A possible explanation for not observing members of the Notch family associated with arteriogenesis upregulated by Sox17 could be a result of differences between the conversion process and EC differentiation, or instability of arteriovenous identity in cultured ECs (Aranguren, X. L. et al., Blood, 122, 3982-3992, (2013)). Nevertheless, the data presented herein show that Sox17 activates a subset of genes induced by Akt-signaling that includes genes associated with morphogenesis and cellular and ECM interactions. A simple model based on the data is that Sox17 activates genes that stabilize barrier and basal matrix interactions while downregulating destabilizers.

Therapeutic replacement of blood vessels has been difficult to establish because so little is known about how ECs might weave themselves into existing vascular infrastructure. Transplanted ECs can improve recovery after injuries and disease and multiple studies in mouse models have shown improved reperfusion after ischemic limb injuries (Li, J. et al., Arteriosclerosis, thrombosis, and vascular biology, 33, 1366-1375, (2013); Han, J. K. et al., Circulation, 130, 1168-1178, (2014); Morita, R. et al., PNAS, 112, 160-165, (2015)). However these findings have not been borne out in clinical studies, as intramuscular delivery of bone marrow-derived cells to patients with peripheral artery disease is ineffective (Mangialardi, G. & Madeddu, P., Curr Diab Rep., 16, 43, 2016)). This discordancy could be due to the differences in injury-type, species, or source cells.

This disclosure defines a mouse-based tractable lineage conversion strategy for engineered ECs and identifies a novel regulator of EC repair functions that could be used to enhance therapeutic EC incorporation into injured vessels. This approach sets forth a platform with which the mechanisms that underlie key EC angiogenic and instructive functions can be reductively tested and identified and translated to the clinical setting.

1. A method of providing endothelial cells, comprising expressing transcription factor Sox17 from an exogenous nucleic acid in reprogramming-derived endothelial cells (rECs).

2. The method of claim 1, wherein said rECs are characterized by expression of surface markers, VE-cadherin, CD31 and VEGFR2, wherein said rECs comprise an exogenously introduced nucleic acid encoding FLI1.

3. The method of claim 1, wherein the rECs are transduced with a vector comprising a nucleic acid encoding Sox17 to achieve expression of Sox17.

4. The method of claim 1, wherein an mRNA encoding Sox17 are delivered into rECs to achieve expression of Sox17.

5. The method of claim 1, wherein Sox17 is expressed constitutively for at least 20 days.

6. The method of claim 1, wherein the Sox17-expressing rECs are cultured for a total duration of at least 28 days.

7. The method of claim 6, wherein the Sox17-expressing rECs are cultured for a total duration of at least 42 days.

8. The method of claim 1, wherein the rECs are derived from non-vascular cells by a process comprising expressing transcription factors ETV2, FLI1 and ERG from exogenous nucleic acids in the non-vascular cells in the presence of a TGFβ signaling inhibitor.

9. The method of claim 8, wherein the expression of ETV2 in the non-vascular cells is transient, and the expressions of FLI and ERG are constitutive.

10. The method of claim 9, wherein ETV2 is transiently expressed for 13-15 days.

11. The method of claim 8, wherein the non-vascular cells are transduced with vectors comprising nucleic acids encoding transcription factors ETV2, FLI1 and ERG to achieve expression of the transcription factors.

12. The method of claim 8, wherein mRNAs encoding the transcription factors ETV2, FLI1 and ERG are delivered into non-vascular cells to achieve expression of the transcription factors.

13. The method of claim 8, wherein the TGFβ signaling inhibitor is present in the cell culture for 20-24 days.

14. The method of claim 13, wherein the TGFβ signaling inhibitor is an inhibitor specific for the type I TGFβ receptors.

15. The method of claim 14, wherein said inhibitor is a polypeptide comprising a soluble form of a type I TGFβ receptor, an antibody directed to a type I TGFβ receptor or ligand, or a small molecule compound.

16. The method of claim 15, wherein said inhibitor is a small molecule compound selected from SB-431542, A 83-01, D 4476, LY 364947, SB 525334, SD 208, and SJN 2511.

17. The method of claim 16, wherein said inhibitor is SB-431542.

18. The method of claim 8, wherein non-vascular cells are cultured for at least 21 days with the expression of ETV2 in the non-vascular cells for the first 13-15 days, the presence of the TGFβ signaling inhibitor for the first 20-21 days, and constitutive expression of FLI1 and ERG.

19. The method of claim 18, wherein the rECs are cultured for a total duration of at least 28 days.

20. The method of claim 18, wherein the rECs are cultured for a total duration of at least 42 days.

21. The method of any one of claim 8, 11 or 18, wherein ERG is ERG1.

22. The method of claim 8, wherein the non-vascular cells are selected from the group consisting of Amniotic Cells (“ACs”), Embryonic Stem (“ES”) cells, induced pluripotent stem cell (iPS cells), mesenchymal stem cells (MSC), myocardial stem cells, myocardial cells, fibroblasts, myoblasts, chondrocytes, hepatocytes, blood cells, epithelial cells and nerve cells.

23. The method of claim 1, wherein the rECs are derived from amniotic cells.

24. A substantially pure population of non-vascular cell-derived ECs, wherein said ECs are characterized by expression of surface markers, VE-cadherin, CD31 and VEGFR2, wherein said ECs comprise an exogenously introduced nucleic acid encoding Sox17.

25. A substantially pure population of non-vascular cell-derived ECs, wherein the ECs are prepared by the method of claim 1.

26. A composition comprising the substantially pure population of ECs of claim 24 and at least one pharmaceutically acceptable carrier or diluents.

27. A method for repairing injured tissue in a human subject, comprising administering to the subject the composition of claim 26 to promote vascularization in said tissue.

28. A method for treating a tumor in a human subject, comprising administering to the subject the composition of claim 26, wherein said ECs are engineered to deliver an anti-tumor agent, and upon administration, said ECs form vessels into said tumor.
